(19) United States
(12) Patent Application Publication
(10) Pub. No.: US 2024/0344024 A1
(43) Pub. Date: Oct. 17, 2024

(54) ENGINEERING OF GAMMA DELTA T CELLS AND COMPOSITIONS THEREOF

(71) Applicant: GAMMADELTA THERAPEUTICS LTD, London (GB)

(72) Inventor: Istvan KOVACS, London (GB)

(21) Appl. No.: 18/294,659

(22) PCT Filed: Aug. 3, 2022
(86) PCT No.: PCT/GB2022/052039

§ 371 Date: Feb. 2, 2024

Related U.S. Application Data
(60) Provisional application No. 63/228,972, filed on Aug. 3, 2021.

(51) Int. Cl. C12N 5/0783; C07K 14/725; C12N 15/86

(52) U.S. Cl. CPC C12N 5/0636; C07K 14/7051; C12N 15/86; C12N 2501/2301; C12N 2501/2304; C12N 2501/2315; C12N 2501/2321; C12N 2501/24; C12N 2740/12022; C12N 2740/15043

(57) ABSTRACT

The present invention provides methods of engineering γδ T cells (e.g., vδ1 T cells and vδ2 T cells) by transduction with a viral vector (e.g., a viral vector with a betaretroviral pseudotype and a 5 Retroviridae family viral vector backbone). Further provided are compositions of engineered γδ T cells and methods of using the same.

### BACKGROUND

The growing interest in T cell immunotherapy for cancer has focused on the evident capacity of engineered T cells as therapeutic moieties. Gamma delta T cells (γδ T cells) represent a subset of T cells that express on their surface a distinct, defining γδ T-cell receptor (TCR). This TCR is made up of one gamma (γ) and one delta (δ) chain. Human γδ T cells can be broadly classified as one or two types: peripheral blood-resident γδ T cells and non-haematopoietic tissue-resident γδ T cells. Most blood-resident γδ T cells express a Vδ2 TCR, whereas this is less common among tissue-resident γδ T cells, which more frequently use Vδ1 and/or other Vδchains.

Relative to αβ T cells, methods for efficient transduction of γδ T cells to express a desired transgene are lacking. Accordingly, there is a need in the field for improved methods for transducing γδ T cells to produce populations of γδ T cells of sufficient quality and quantity for use as therapies, e.g., as adoptive T cell therapies.

### SUMMARY OF THE INVENTION

In one aspect, the invention features a method of producing a population of engineered γδ T cells by transducing a population of γδ T cells with a viral vector having a betaretroviral pseudotype and a Retroviridae family viral vector backbone. The betaretroviral pseudotype may be baboon endogenous virus (BaEV). The betaretroviral pseudotype may be RD114.

In some embodiments, the Retroviridae family viral vector backbone is a retroviral vector backbone (e.g., lentiviral backbone, gammaretroviral backbone, or alpharetroviral backbone).

The engineered γδ T cells may be Vδ1 T cells. The engineered γδ T cells may be Vδ2 T cells. The engineered γδ T cells may be non-Vδ1/Vδ2 T cells.

In some embodiments, the viral vector includes a transgene. The transgene may encode a cell surface receptor (e.g., a chimeric antigen receptor (CAR)) and/or a cytokine (e.g., a secreted cytokine or a membrane-bound cytokine). In some embodiments, the transgene encodes IL-15 (e.g., secreted IL-15 or membrane-bound IL-15). In some embodiments, the viral vector includes a first transgene and a second transgene. In some embodiments, the first transgene encodes a CAR, and the second transgene encodes an armor protein (e.g., a cytokine, e.g., IL-15, e.g., secreted IL-15 or membrane-bound IL-15).

In some embodiments, the CAR targets CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gpl20, HIV-1 envelope glycoprotein gp41, GD2, CD123, CD33, CD138, CD23, CD30, CD56, c-Met, mesothelin, GD3, HERV-K, IL-IIRalpha, kappa chain, lambda chain, CSPG4, ERBB2, EGFRvIII, VEGFR2, HER2-HER3 in combination, HER1-HER2 in combination, NY-ESO-1, synovial sarcoma X breakpoint 2 (SSX2), melanoma antigen (MAGE), melanoma antigen recognized by T cells 1 (MART-1), gp100, prostate specific antigen (PSA), prostate specific membrane antigen (PSMA), prostate stem cell antigen (PSCA), g9d2, or a combination thereof.

In another aspect, the invention features a method of producing a population of engineered γδ T cells. The method includes providing a starting population of γδ T cells and culturing the starting population of γδ T cells for a first culture period in the absence of a viral vector to produce a population of primed γδ T cells. The method may further include culturing the population of primed γδ T cells for a second culture period in the presence of a viral vector having a betaretroviral pseudotype in an amount effective to transduce at least 3% (e.g., at least 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the primed γδ T cells, thereby producing the population of engineered γδ T cells.

In some embodiments, the viral vector is in an amount effective to transduce at least 20% of the primed γδ T cells.

In some embodiments, the first culture period is for 1 day or longer (e.g., 1 day, 2 days, 3 days, 4 days, 5 days, 6 days, 7 days, 8 days, 9 days, 10 days, or longer, e.g., 1-3 days, 3-5 days, 5-7 days, 7-10 days, or longer). In some embodiments, the first culture period is for 2 days or longer (e.g., 2 days, 3 days, 4 days, 5 days, 6 days, 7 days, 8 days, 9 days, 10 days, or longer, e.g., 1-3 days, 3-5 days, 5-7 days, 7-10 days, or longer). In some embodiments, the first culture period is for 5 days or longer (e.g., 5 days, 6 days, 7 days, 8 days, 9 days, 10 days, or longer, e.g., 5-7 days, 7-10 days, or longer). In some embodiments, the first culture period is for 7 days or longer (e.g., 7 days, 8 days, 9 days, 10 days, or longer, e.g., 7-10 days, or longer).

In some embodiments, the second culture period is for 2 days or longer (e.g., 2 days, 3 days, 4 days, 5 days, 6 days, 7 days, 8 days, 9 days, 10 days, 11 days, 12 days, 13 days, 14 days, or longer, e.g., 2-4 days, 4-7 days, 7-10 days, 10-14 days, or longer). In some embodiments, the second culture period is for 7 days or longer (e.g., 8 days, 9 days, 10 days, 11 days, 12 days, 13 days, 14 days, or longer, e.g., 7-10 days, 10-14 days, or longer).

In some embodiments, the population of primed γδ T cells expresses ASCT-1 and/or ASCT-2. In some embodiments, the population of primed γδ T cells lacks functional expression of a VSV-G entry receptor (e.g., an LDL receptor).

In some embodiments, the viral vector is cultured with the primed γδ T cells at a multiplicity of infection (MOI) no greater than 10 (e.g., no greater than 5, e.g., from about 1 to about 5).

In another aspect, the invention features a method of producing a population of engineered γδ T cells by providing a starting population of γδ T cells; and culturing the starting population of γδ T cells in the presence of IL-15 and a viral vector having a betaretroviral pseudotype in an amount effective to transduce at least 3% (e.g., at least 4%, 5%, 6%, 7%, 8%, 9%, 10%, 11%, 12%, 13% m 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the starting population of γδ T cells, thereby producing the population of engineered γδ T cells.

In some embodiments, the starting population of γδ T cells lack expression of ASCT-1 and/or ASCT-2. In some embodiments, the population of engineered γδ T cells expresses ASCT-1 and/or ASCT-2. The starting population of γδ T cells may lack functional expression of a VSV-G entry receptor (e.g., an LDL receptor).

In some embodiments, the viral vector is cultured with the starting population of γδ T cells at an MOI no greater than 10 (e.g., no greater than 5, e.g., from about 1 to about 5).

In some embodiments, the viral vector has a betaretroviral pseudotype of BaEV or RD114.

In some embodiments, the viral vector includes a Retroviridae family viral vector backbone. The Retroviridae family viral vector backbone may be a retroviral vector backbone (e.g., lentiviral backbone, gammaretroviral backbone, or alpharetroviral backbone).

The engineered γδ T cells may be Vδ1 T cells. The engineered γδ T cells may be Vδ2 T cells. The engineered γδ T cells may be non-Vδ1/Vδ2 T cells.

In some embodiments, the viral vector includes a transgene. The transgene may encode a cell surface receptor, e.g., a chimeric antigen receptor (CAR) and/or a cytokine (e.g., a secreted cytokine or a membrane-bound cytokine). In some embodiments, the transgene encodes IL-15 (e.g., secreted IL-15 or membrane-bound IL-15). In some embodiments, the viral vector includes a first transgene and a second transgene. In some embodiments, the first transgene encodes a CAR, and the second transgene encodes an armor protein (e.g., a cytokine, e.g., IL-15, e.g., secreted IL-15 or membrane-bound IL-15).

In some embodiments, the CAR targets CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gpl20, HIV-1 envelope glycoprotein gp41, GD2, CD123, CD33, CD138, CD23, CD30, CD56, c-Met, mesothelin, GD3, HERV-K, IL-IIRalpha, kappa chain, lambda chain, CSPG4, ERBB2, EGFRvIII, VEGFR2, HER2-HER3 in combination, HER1-HER2 in combination, NY-ESO-1, SSX2, MAGE, MART-1, gp100, PSA, PSMA, PSCA, g9d2, or a combination thereof.

In another aspect, the invention features a method of producing a population of γδ T cells expressing a CAR by transducing a population of γδ T cells with a viral vector that includes a transgene encoding the CAR; a betaretroviral pseudotype; and a Retroviridae family viral vector backbone.

In another aspect, the invention features a method of producing a population of γδ T cells expressing a CAR and an armor protein by transducing a population of γδ T cells with a viral vector that includes a first transgene encoding the CAR; a second transgene encoding the armor protein; a betaretroviral pseudotype; and a Retroviridae family viral vector backbone. In some embodiments, the armor protein is a cytokine (e.g., a membrane-bound cytokine or a secreted cytokine (e.g., membrane-bound IL-15 or secreted IL-15).

In some embodiments, the betaretroviral pseudotype is BaEV. In other embodiments, the betaretroviral pseudotype is RD114.

In some embodiments, the viral vector includes a Retroviridae family viral vector backbone. The Retroviridae family viral vector backbone may be a retroviral vector backbone (e.g., lentiviral backbone, gammaretroviral backbone, or alpharetroviral backbone).

The γδ T cells may be Vδ1 T cells. The γδ T cells may be Vδ2 T cells. The γδ T cells may be non-Vδ1/Vδ2 T cells.

In another aspect, the invention features a method of producing a population of γδ T cells expressing a CAR by providing a starting population of γδ T cells and culturing the starting population of γδ T cells for a first culture period in the absence of a viral vector to produce a population of primed γδ T cells. The method may further include culturing the population of primed γδ T cells for a second culture period in the presence of a viral vector having a betaretroviral pseudotype and a transgene encoding the CAR, wherein the viral vector is in an amount effective to transduce at least 3% (e.g., at least 4%, 5%, 6%, 7%, 8%, 9%, 10%, 11%, 12%, 13% m 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the primed γδ T cells, thereby producing the population of γδ T cells expressing the CAR.

In another aspect, the invention features a method of producing a population of γδ T cells expressing a CAR and an armor protein by providing a starting population of γδ T cells and culturing the starting population of γδ T cells for a first culture period in the absence of a viral vector to produce a population of primed γδ T cells. The method may further include culturing the population of primed γδ T cells for a second culture period in the presence of a viral vector having a betaretroviral pseudotype, a first transgene encoding the CAR, and a second transgene encoding the armor protein, wherein the viral vector is in an amount effective to transduce at least 3% (e.g., at least 4%, 5%, 6%, 7%, 8%, 9%, 10%, 11%, 12%, 13% m 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the primed γδ T cells, thereby producing the population of γδ T cells expressing the CAR and the armor protein. In some embodiments, the transgene encodes IL-15 (e.g., secreted IL-15 or membrane-bound IL-15). In some embodiments, the viral vector includes a first transgene and a second transgene. In some embodiments, the first transgene encodes a CAR, and the second transgene encodes an armor protein (e.g., a cytokine, e.g., IL-15, e.g., secreted IL-15 or membrane-bound IL-15).

In some embodiments, the viral vector is in an amount effective to transduce at least 20% of the primed γδ T cells.

In some embodiments, the first culture period is for 1 day or longer (e.g., 1 day, 2 days, 3 days, 4 days, 5 days, 6 days, 7 days, 8 days, 9 days, 10 days, or longer, e.g., 1-3 days, 3-5 days, 5-7 days, 7-10 days, or longer). In some embodiments, the first culture period is for 2 days or longer (e.g., 2 days, 3 days, 4 days, 5 days, 6 days, 7 days, 8 days, 9 days, 10 days, or longer, e.g., 1-3 days, 3-5 days, 5-7 days, 7-10 days, or longer). In some embodiments, the first culture period is for 5 days or longer (e.g., 5 days, 6 days, 7 days, 8 days, 9 days, 10 days, or longer, e.g., 5-7 days, 7-10 days, or longer). In some embodiments, the first culture period is for 7 days or longer (e.g., 7 days, 8 days, 9 days, 10 days, or longer, e.g., 7-10 days, or longer).

In some embodiments, the second culture period is for 2 days or longer (e.g., 2 days, 3 days, 4 days, 5 days, 6 days, 7 days, 8 days, 9 days, 10 days, 11 days, 12 days, 13 days, 14 days, or longer, e.g., 2-4 days, 4-7 days, 7-10 days, 10-14 days, or longer). In some embodiments, the second culture period is for 7 days or longer (e.g., 8 days, 9 days, 10 days, 11 days, 12 days, 13 days, 14 days, or longer, e.g., 7-10 days, 10-14 days, or longer).

In some embodiments, the population of primed γδ T cells expresses ASCT-1 and/or ASCT-2. In some embodiments, the population of primed γδ T cells lacks functional expression of a VSV-G entry receptor (e.g., an LDL receptor). In some embodiments, more than 95% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 96% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 97% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 98% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 99% of the population of primed γδ T cells lacks a sufficient level of a VSV-G entry receptor expression (e.g., LDL receptor) to mediate detectable VSV-G entry (e.g., as measured by BlaM-Vpr-based assay).

In some embodiments, the viral vector is cultured with the primed γδ T cells at an MOI no greater than 10 (e.g., no greater than 5, e.g., from about 1 to about 5).

In another aspect, the invention features a method of producing a population of γδ T cells expressing a CAR by providing a starting population of γδ T cells; and culturing the starting population of γδ T cells in the presence of IL-15 and a viral vector having a betaretroviral pseudotype and a transgene encoding the CAR, wherein the viral vector is in an amount effective to transduce at least 3% (e.g., at least 4%, 5%, 6%, 7%, 8%, 9%, 10%, 11%, 12%, 13% m 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the starting population of γδ T cells, thereby producing the population of engineered γδ T cells expressing the CAR.

In another aspect, the invention features a method of producing a population of γδ T cells expressing a CAR and an armor protein by providing a starting population of γδ T cells; and culturing the starting population of γδ T cells in the presence of IL-15 and a viral vector having a betaretroviral pseudotype, a first transgene encoding the CAR, and a second transgene encoding an armor protein, wherein the viral vector is in an amount effective to transduce at least 3% (e.g., at least 4%, 5%, 6%, 7%, 8%, 9%, 10%, 11%, 12%, 13% m 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the starting population of γδ T cells, thereby producing the population of engineered γδ T cells expressing the CAR and the armor protein. In some embodiments, the armor protein is a cytokine, e.g., IL-15, e.g., secreted IL-15 or membrane-bound IL-15.

In some embodiments, the starting population of γδ T cells lack expression of ASCT-1 and/or ASCT-2. The population of engineered γδ T cells may express ASCT-1 and/or ASCT-2. The starting population of γδ T cells may lack functional expression of a VSV-G entry receptor (e.g., an LDL receptor). In some embodiments, more than 95% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 96% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 97% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 98% of the population of primed γδ T cells lacks a sufficient level of a VSV-G entry receptor expression (e.g., LDL receptor) to mediate detectable VSV-G entry (e.g., as measured by BlaM-Vpr-based assay). In some embodiments, more than 99% of the population of primed γδ T cells lacks a sufficient level of a VSV-G entry receptor expression (e.g., LDL receptor) to mediate detectable VSV-G entry (e.g., as measured by BlaM-Vpr-based assay).

In some embodiments, the viral vector is cultured with the starting population of γδ T cells at an MOI no greater than 10 (e.g., no greater than 5, e.g., from about 1 to about 5).

In some embodiments, the betaretroviral pseudotype is BaEV or RD114.

In some embodiments, the viral vector includes a Retroviridae family viral vector backbone. The Retroviridae family viral vector backbone may be a retroviral vector backbone (e.g., lentiviral backbone, gammretroviral backbone, or alpharetroviral backbone).

The engineered γδ T cells may be Vδ1 T cells. The engineered γδ T cells may be Vδ2 T cells. The engineered γδ T cells may be non-Vδ1/Vδ2 T cells.

In some embodiments, the CAR targets CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gpl20, HIV-1 envelope glycoprotein gp41, GD2, CD123, CD33, CD138, CD23, CD30, CD56, c-Met, mesothelin, GD3, HERV-K, IL-IIRalpha, kappa chain, lambda chain, CSPG4, ERBB2, EGFRvIII, VEGFR2, HER2-HER3 in combination, HER1-HER2 in combination, NY-ESO-1, SSX2, MAGE, MART-1, gp100, PSA, PSMA, PSCA, g9d2, or a combination thereof.

In another aspect, the invention features a population of engineered γδ T cells produced by a method as described herein.

In some embodiments, at least 10% (e.g., at least 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the population expresses a CAR. In some embodiments, at least 50% (e.g., at least 55%, 60%, 65%, 70%, 75%, 80%, 85%, 90%, 95%, 97%, 99%, or substantially all) of the population of engineered γδ T cells expresses a CAR. In some embodiments, at least 10% (e.g., at least 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the population of engineered γδ T cells expresses an armor protein, e.g., a cytokine (e.g., a secreted cytokine or a membrane-bound cytokine (e.g., IL-15, e.g., secreted IL-15 or membrane-bound IL-15). In some embodiments, at least 50% (e.g., at least 55%, 60%, 65%, 70%, 75%, 80%, 85%, 90%, 95%, 97%, 99%, or substantially all) of the population of engineered γδ T cells expresses an armor protein, e.g., a cytokine (e.g., a secreted cytokine or a membrane-bound cytokine (e.g., IL-15, e.g., secreted IL-15 or membrane-bound IL-15). In some embodiments, at least 10% (e.g., at least 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the population of engineered γδ T cells expresses a CAR and an armor protein, e.g., a cytokine (e.g., a secreted cytokine or a membrane-bound cytokine (e.g., IL-15, e.g., secreted IL-15 or membrane-bound IL-15). In some embodiments, at least 50% (e.g., at least 55%, 60%, 65%, 70%, 75%, 80%, 85%, 90%, 95%, 97%, 99%, or substantially all) of the population of engineered γδ T cells expresses a CAR and an armor protein, e.g., a cytokine (e.g., a secreted cytokine or a membrane-bound cytokine (e.g., IL-15, e.g., secreted IL-15 or membrane-bound IL-15).

In another aspect, the invention features a population of γδ T cells expressing a CAR produced by a method as described herein.

In another aspect, the invention features a population of γδ T cells expressing a CAR and an armor protein produced by a method as described herein. In some embodiments, the armor protein is a cytokine (e.g., a secreted cytokine or a membrane-bound cytokine (e.g., IL-15, e.g., secreted IL-15 or membrane-bound IL-15).

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. In some instances, “about” encompass variations of +20%, in some instances +10%, in some instances +5%, in some instances +1%, or in some instances +0.1% from the specified value, as such variations are appropriate to perform the disclosed methods.

As used herein, the term “engineered γδ T cell” refers to a γδ T cell that expresses a transgene (i.e., a gene that has been transduced into the engineered γδ T cell or a parental cell thereof).

As used herein, the term “primed γδ T cell” refers to a starting population (e.g., an endogenous population of γδ T cells) that has been affected by a culture condition. In some instances, a primed γδ T cell has a different functional viral entry receptor profile relative to its unprimed counterpart before experiencing the culture condition. In some embodiments, a population of primed γδ T cells is an expanded population of γδ T cells.

As used herein, an “expanded population of γδ cells” refers to a population of haematopoietic cells including γδ T cells that has been cultured in a condition and for a duration that has induced the expansion of γδ cells, i.e., increased γδ cell number. Likewise, an “expanded population of Vδ1 T cells,” as used herein, refers to a population of haematopoietic cells including Vδ1 T cells that has been cultured in a condition and for a duration that has induced the expansion of Vδ1 T cells, i.e., increased Vδ1 cell number. Similarly, an “expanded population of Vδ2 T cells,” as used herein, refers to a population of haematopoietic cells including Vδ2 T cells that has been cultured in a condition and for a duration that has induced the expansion of Vδ2 T cells, i.e., increased Vδ2 cell number.

As used herein, a “population” of γδ T cells refers to a group of three or more γδ T cells (e.g., at least 10, at least 10², at least 10³, at least 10⁴, at least 10⁵, at least 10⁶, at least 10⁷, at least 10⁸, at least 10⁹, at least 10¹⁰, at least 10¹¹, at least 10¹², or at least 10¹³) γδ T cells (e.g., engineered γδ T cells). A population of a particular cell type (e.g., a population of endogenous γδ T cells, a population of primed γδ T cells, or a population of engineered γδ T cells) refers to the cells of that type and not to cells of a different type within a broader population. For example, if 10% of the cells of a starting population of 10⁸ T cells are γδ T cells, the starting population of γδ T cells is 10⁷.

As used herein, an “armor protein” refers to a protein encoded by a transgene that, when expressed by a γδ T cell (e.g., a γδ T cell expressing a CAR), increases persistent or increased immunogenicity of the γδ T cell toward a target cell, e.g., through paracrine signaling (e.g., cytokine signaling) to improve, e.g., cell persistence, cell viability, activation and other desired characteristics. An armor protein can be a membrane-bound protein or a soluble protein. For example, armor proteins include membrane-bound proteins, such as a membrane-bound receptor (e.g., as TCR, a natural cytotoxicity receptor (e.g., NKp30, NKp44, or NKp46), a cytokine receptor (e.g., IL-12 receptor), and/or a chemokine receptor (e.g., CCR2 receptor) and/or a membrane-bound ligand or cytokine (e.g., membrane-bound IL-15, membrane-bound IL-7, membrane-bound CD40L, membrane-bound 4-1BB, membrane-bound 4-1BBL, membrane bound CCL19). Additionally, or alternatively, armor proteins can be soluble proteins, such as soluble ligands or cytokines (e.g., soluble IL-15, soluble IL-7, soluble IL-12, soluble CD40L, soluble 4-1BBL, and/or soluble CCL19). In some embodiments, an armor protein is not antigen specific.

As used herein, “IL-15” refers to native or recombinant IL-15 or a variant thereof that acts as an agonist for one or more IL-15 receptor (IL-15R) subunits (e.g., mutants, muteins, analogues, subunits, receptor complexes, fragments, isoforms, and peptidomimetics thereof). IL-15, like IL-2, is a known T-cell growth factor that can support proliferation of an IL-2-dependent cell line, CTLL-2. IL-15 was first reported by Grabstein et al. (Science 264.5161: 965-969, 1994) as a 114-amino acid mature protein. The term “IL-15,” as used herein, means native or recombinant IL-15 and muteins, analogs, subunits thereof, or complexes thereof (e.g., receptor complexes, e.g., sushi peptides, as described in PCT Pub. No. WO 2007/046006), and each of which can stimulate proliferation of CTLL-2 cells. In the CTLL-2 proliferation assays, supernatants of cells transfected with recombinantly expressed precursor and in-frame fusions of mature forms of IL-15 can induce CTLL-2 cell proliferation.

Human IL-15 can be obtained according to the procedures described by Grabstein et al. (Science 264.5161: 965-969, 1994) or by conventional procedures such as polymerase chain reaction (PCR). A deposit of human IL-15 cDNA was made with the ATCC® on Feb. 19, 1993, and assigned accession number 69245.

The amino acid sequence of human IL-15 (Gene ID 3600) is found in Genbank under accession locator NP000576.1 GI: 10835153 (isoform 1) and NP_751915.1 GI: 26787986 (isoform 2). The murine (Mus musculus) IL-15 amino acid sequence (Gene ID 16168) is found in Genbank under accession locator NP_001241676.1 GI: 363000984.

IL-15 can also refer to IL-15 derived from a variety of mammalian species, including, for example, human, simian, bovine, porcine, equine, and murine. An IL-15 “mutein” or “variant”, as referred to herein, is a polypeptide substantially homologous to a sequence of a native mammalian IL-15 but that has an amino acid sequence different from a native mammalian IL-15 polypeptide because of an amino acid deletion, insertion, or substitution. Variants may comprise conservatively substituted sequences, meaning that a given amino acid residue is replaced by a residue having similar physiochemical characteristics. Examples of conservative substitutions include substitution of one aliphatic residue for another, such as lie, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-15 variants are also encompassed by the invention. Examples of such variants are proteins that result from alternate mRNA splicing events or from proteolytic cleavage of the IL-15 protein, wherein the IL-15 binding property is retained. Alternate splicing of mRNA may yield a truncated but biologically active IL-15 protein. Variations attributable to proteolysis include, for example, differences in the N- or C-termini upon expression in different types of host cells, due to proteolytic removal of one or more terminal amino acids from the IL-15 protein (generally from 1-10 amino acids). In some embodiments, the terminus of the protein can be modified to alter its physical properties, for example, with a chemical group such as polyethylene glycol (Yang et al. Cancer 76:687-694, 1995). In some embodiments, the terminus or interior of the protein can be modified with additional amino acids (Clark-Lewis et al. PNAS 90:3574-3577, 1993).

As used herein, “non-haematopoietic cells” include stromal cells and epithelial cells. Stromal cells are non-haematopoietic connective tissue cells of any organ and support the function of the parenchymal cells of that organ. Examples of stromal cells include fibroblasts, pericytes, mesenchymal cells, keratinocytes, endothelial cells, and non-hematological tumor cells. Epithelial cells are non-haematopoietic cells that line the cavities and surfaces of blood vessels and organs throughout the body. They are normally squamous, columnar, or cuboidal in shape and can be arranged as a single layer of cells, or as layers of two or more cells.

As used herein, “non-haematopoietic tissue-resident γδ T cells,” “non-haematopoietic tissue-derived,” and “non-haematopoietic tissue-native γδ T cells” refer to γδ T cells that were present in a non-haematopoietic tissue at the time the tissue is explanted. Non-haematopoietic tissue-resident γδ T cells may be obtained from any suitable human or non-human animal non-haematopoietic tissue. Non-haematopoietic tissue is a tissue other than blood or bone marrow. In some embodiments, the γδ T cells are not obtained from particular types of samples of biological fluids, such as blood or synovial fluid. Examples of such suitable human or non-human animal non-haematopoietic tissues include skin or a portion thereof (e.g., dermis or epidermis), the gastrointestinal tract (e.g., gastrointestinal epithelium, colon, small intestine, stomach, appendix, cecum, or rectum), mammary gland tissue, lung (preferably wherein the tissue is not obtained by bronchoalveolar lavage), prostate, liver, and pancreas. In some embodiments, non-haematopoietic tissue-resident γδ T cells can be derived from a lymphoid tissue, such as thymus, spleen, or tonsil. The γδ T cells may also be resident in human cancer tissues, e.g., breast and prostate. In some embodiments, the γδ T cells are not obtained from human cancer tissue. Non-haematopoietic tissue samples may be obtained by standard techniques e.g., by explant (e.g., biopsy). Non-haematopoietic tissue-resident γδ T cells include e.g., Vδ1 T cells, double negative (DN) T cells, Vδ2 T cells, Vδ3 T cells, and Vδ5 T cells.

As used herein, the phrase “in an amount effective to” refers to an amount that induces a detectable result (e.g., a number of cells having a statistically significant increased number relative to its starting population, e.g., at a p<0.05).

As used herein, an “expanded population of γδ cells” refers to a population of haematopoietic cells including γδ T cells that has been cultured in a condition and for a duration that has induced the expansion of γδ cells, i.e., increased γδ cell number. Likewise, an “expanded population of Vδ1 T cells,” as used herein, refers to a population of haematopoietic cells including Vδ1 T cells that has been cultured in a condition and for a duration that has induced the expansion of Vδ1 T cells, i.e., increased Vδ1 cell number. Similarly, an “expanded population of Vδ2 T cells,” as used herein, refers to a population of haematopoietic cells including Vδ2 T cells that has been cultured in a condition and for a duration that has induced the expansion of Vδ2 T cells, i.e., increased Vδ2 cell number

The term “marker” herein to refers to a DNA, RNA, protein, carbohydrate, glycolipid, or cell-based molecular marker, the expression or presence of which in a patient's sample can be detected by standard methods (or methods disclosed herein).

A cell or population of cells that “expresses” a marker of interest is one in which mRNA encoding the protein, or the protein itself, including fragments thereof, is determined to be present in the cell or the population. Expression of a marker can be detected by various means. For example, in some embodiments, expression of a marker refers to a surface density of the marker on a cell. Mean fluorescence intensity (MFI), for example, as used as a readout of flow cytometry, is representative of the density of a marker on a population of cells. A person of skill in the art will understand that MFI values are dependent on staining parameters (e.g., concentration, duration, and temperature) and fluorochrome composition. However, MFI can be quantitative when considered in the context of appropriate controls. For instance, a population of cells can be said to express a marker if the MFI of an antibody to that marker is significantly higher than the MFI of an appropriate isotype control antibody on the same population of cells, stained under equivalent conditions. Additionally, or alternatively, a population of cells can be said to express a marker on a cell-by-cell basis using a positive and negative gate according to conventional flow cytometry analytical methods (e.g., by setting the gate according to isotype or “fluorescence-minus-one” (FMO) controls). By this metric, a population can be said to “express” a marker if the number of cells detected positive for the marker is significantly higher than background (e.g., by gating on an isotype control).

As used herein, “functional expression of a VSV-G entry receptor” refers to a level of VSV-G entry receptor expression sufficient to mediate detectable VSV-G entry in at least 5% of the target population of cells, as measured by a beta-lactamase-Vpr (BIaM-VpR)-based assay. See, e.g., Cavrois et al., Nat Biotechnol. 11:1151-1154, 2002. Conversely, in a population of cells that “lacks functional expression of a VSV-G entry receptor,” more than 95% of the cell population lacks the sufficient level of VSV-G entry receptor expression to mediate detectable VSV-G entry, as measured by a BIaM-VpR-based assay.

As used herein, when a population's expression is stated as a percentage of positive cells and that percentage is compared to a corresponding percentage of positive cells of a reference population, the percentage difference is a percentage of the parent population of each respective population. For example, if a marker is expressed on 10% of the cells of population A, and the same marker is expressed on 1% of the cells of population B, then population A is said to have a 9% greater frequency of marker-positive cells than population B (i.e., 10%-1%, not 10%+1%). When a frequency is multiplied through by the number of cells in the parent population, the difference in absolute number of cells is calculated. In the example given above, if there are 100 cells in population A, and 10 cells in population B, then population A has 100-fold the number of cells relative to population B, i.e., (10%×100)+(1%×10).

An expression level of a marker may be a nucleic acid expression level (e.g., a DNA expression level or an RNA expression level, e.g., an mRNA expression level). Any suitable method of determining a nucleic acid expression level may be used. In some embodiments, the nucleic acid expression level is determined using qPCR, rtPCR, RNA-seq, multiplex qPCR or RT-qPCR, microarray analysis, serial analysis of gene expression (SAGE), MASSARRAY® technique, in situ hybridization (e.g., FISH), or combinations thereof.

As used herein, a “reference population” of cells refers to a population of cells corresponding to the cells of interest, against which a phenotype of the cells of interest are measured. For example, a level of expression of a marker on a separated population of non-haematopoietic tissue-derived γδ cells may be compared to the level of expression of the same marker on a haematopoietic tissue-derived γδ T cell (e.g., a blood-resident γδ cell, e.g., a blood-resident γδ cell derived from the same donor or a different donor) or a non-haematopoietic tissue-derived γδ T cell expanded under different conditions (e.g., in the presence of substantial TCR activation, in the presence of an exogenous TCR activation agent (e.g., anti-CD3), or in substantial contact with stromal cells (e.g., fibroblasts)). A population may also be compared to itself at an earlier state. For example, a reference population can be a separated cell population prior to its expansion. In this case, the expanded population is compared to its own composition prior to the expansion step, i.e., its past composition, in this case, is the reference population.

As used herein, the term “chimeric antigen receptor” or alternatively a “CAR” refers to a recombinant polypeptide construct including an extracellular antigen binding domain, a transmembrane domain, and, optionally, an intracellular domain that propagates an activation signal that activates the cell and/or a costimulatory signal. In some embodiments, the CAR includes an optional leader sequence at the N-terminus of the CAR fusion protein.

### BRIEF DESCRIPTION OF THE DRAWINGS

FIGS. 1A and 1B are graphs showing broad tropism VSV-G pseudotyped lentiviral vectors cannot transduce Vδ1 γδ T cells. Representative dot plots show γδ T cells transduced with VSV-G (FIG. 1A) or BaEV (FIG. 1B) pseudotyped GFP encoding lentiviral vectors using various multiplicity of infections at day 7 of the expansion culture. Transduction efficiency was determined by FACS analysis 72 hours post-transduction. UTD, untransduced controls; MOI, multiplicity of infection; NVP, nevirapine (RT inhibitor).

FIGS. 2A and 2B are graphs showing ransduction of Vδ1 γδ T-cells with VSV-G pseudotyped CAR encoding lentiviral vectors result in pseudotransduction. FIG. 2A shows representative dot plots of CAR+ve Vδ1 γδ T-cells 4 (top row) or 8 (bottom row) days after transduction with VSV-G pseudotyped CAR encoding lentiviral vectors at MOI=1 in the presence or absence of nevirapine. FIG. 2B is a graph showing percentage of CAR+ve Vδ1 γδ T-cells 4 (black bars) or 8 (dotted bars) days after transduction with VSV-G pseudotyped CAR encoding lentiviral vectors at various MOIs (MOI=5-0.1) in the presence or absence of nevirapine. UTD, untransduced controls; MOI, multiplicity of infection; CAR, chimeric antigen receptor; NVP, nevirapine.

FIGS. 3A and 3B are graphs showing that cytokine priming is a major determinant of Vδ1 γδ T cells transduction by BaEV pseudotyped lentiviral vectors. FIG. 3A is a bar graph showing the percentage of GFP+ve Vδ1 cells transduced at MOI=1 with GFP encoding BaEV pseudotyped lentiviral vector three days post-transduction. Cells were transduced at the start of the culture (day 0) or at day7, day 10, day 14 and day 15 of the expansion phase. FIG. 3B shows representative dot plots of cells transduced at day 14 of the expansion culture. UTD, untransduced controls; MOI, multiplicity of infection; GFP, green fluorescent protein; NVP, nevirapine.

FIGS. 4A and 4B are graphs showing transduction efficiency of Vδ1 γδ T cells correlates with the multiplicity of infection (MOI). FIG. 4A shows percentage of CAR+ve Vδ1 cells 3 days after transduction with CAR encoding BaEV pseudotyped lentiviral vectors with different MOIs. Cells were transduced on day 10 of the expansion. FIG. 4B shows representative dot plot shows CAR+ve cells transduced at MOI=5. UTD, untransduced controls; MOI, multiplicity of infection; CAR, chimeric antigen receptor; NVP, nevirapine.

FIGS. 5A and 5B are graphs showing BaEV pseudotyped lentiviral vectors transduce both Vδ1 and non-Vδ1 (Vδ2, Vδ3) γδ T-cells. Dot plots show CAR (FIG. 5A) and GFP (FIG. 5B) expressing Vδ1 and non-Vδ1 (Vδ2, Vδ3) γδ T-cells. Cells were transduced with BaEV pseudotyped vectors (MOI=5) and transduction efficiencies were determined three days post transduction by gating on pan-γδ T cells, followed by gating on Vδ1 cells.

FIG. 6 is a set of graphs showing transduction of Vδ1 γδ T cells with BaEV pseudotyped lentiviral vectors can be further enhanced by repeated transductions. Vδ1 cells were transduced at MOI=1 with BaEV pseudotyped CAR encoding lentiviral vectors either on day 10 (1 hit) or on two consecutive days (2 hits: day 10 and 11). Percentage of CAR+ve cells were determined 72 hours after transduction.

FIG. 7 is a graph showing transduction in the presence of vectofusin is as efficient as in the presence of retronectin. Vδ1 cells were transduced in the presence of retronectin (left) or vectofusin (right) with various MOIs and various frequencies (one or two hit). Cells were transduced on day 10 of the expansion and FACS analysis was conducted three days post-transduction.

FIG. 8 is a set of graphs showing Vδ1 cells can be transduced with RD114 pseudotyped viral vectors. Vδ1 cells were transduced at MOI=1 with BaEV pseudotyped CAR encoding lentiviral or RD114 pseudotyped gammaretroviral vectors. Dot plots show CAR expressing Vδ1 cells three days after transductions.

### DETAILED DESCRIPTION

The present invention provides methods of engineering γδ T cells (e.g., vδ1 T cells and vδ2 T cells) by transduction with a viral vector (e.g., a viral vector with a betaretroviral pseudotype and a Retroviridae family viral vector backbone). Further provided are compositions of engineered γδ T cells and methods of using the same.

The present invention is based, in part, on the unexpected discovery that γδ T cells can be transduced with a betaretroviral pseudotyped viral vector to a high level. Relative to other lymphocyte types, γδ T cells are non-permissive for retroviral transductions, e.g., using a VSV-G pseudotyped viral vector. VSV-G vectors easily transduce αβ T cells as well as NK cells, which are the closest cell types to γδ T cells. Thus, it was not expected that a betaretroviral pseudotyped viral vector would be able to transduce γδ T cells. Furthermore, the present invention also based on the discovery of optimal culture conditions and durations of γδ T cells in the presence of a viral vector in order to transduce a population γδ T cells with the vector. The methods of transduction described herein allow efficient transduction of γδ T cells in order to produce an engineered population of γδ T cells expressing a desired transgene.

### Methods of Transduction

In one aspect, the invention provides a method for producing a population of engineered γδ T cells by transducing a population of γδ T cells (e.g., Vδ1 T cells, Vδ2 T cells, and/or non-Vδ1/Vδ2 T cells) with a viral vector that includes a betaretroviral pseudotype and a Retroviridae family (e.g., retroviral) vector backbone. The retroviral vector backbone may be, e.g., a lentiviral backbone, a gammaretroviral backbone, or an alpharetroviral backbone. The betaretroviral psuedotype may be, e.g., BaEV or RD114. In some embodiments the betaretroviral psuedotype is BaEV. In some embodiments the betaretroviral psuedotype is RD114.

In another aspect, the invention provides a method of producing a population of engineered γδ T cells by providing a starting population of γδ T cells, priming the γδ T cells in the absence of a viral vector, and culturing the population of primed γδ T cells in the presence of a viral vector in an amount effective to transduce at least 3% (e.g., at least 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the primed γδ T cells. In some embodiments, the population of primed γδ T cells is cultured in the presence of a viral vector in an amount effective to transduce at least 5% of the primed γδ T cells. In some embodiments, the population of primed γδ T cells is cultured in the presence of a viral vector in an amount effective to transduce at least 20% of the primed γδ T cells.

The primed γδ T cells may be obtained by culturing the starting population of γδ T cells in the absence of a viral vector. For example, the starting population of γδ T cells may be cultured for a first culture period of at least 1 hour (e.g., at least 2 hours, 3 hours, 4 hours, 5 hours, 6 hours, 12 hours, 1 day, 2 days, 3 days, 4 days, 5 days, 6 days, 7 days, 8 days, 9 days, 10 days, 11 days, 12 days, 13 days, 14 days, or longer, e.g., from about 1 hour to about 14 days, from about 6 hours to about 14 days, from about 1 day to about 14 days, from about 2 days to about 14 days, from about 5 days to about 14 days, from about 7 days to about 14 days, from about 5 days to about 10 days, from about 5 days to about 7 days, or from about 7 days to about 10 days). When the primed γδ T cells are obtained, e.g., following culturing of the cells in the absence of a viral vector, the primed γδ T cells may be further cultured for a second culture period of at least 1 day (e.g., at least 2 days, 3 days, 4 days, 5 days, 6 days, 7 days, 8 days, 9 days, 10 days, 11 days, 12 days, 13 days, 14 days, or longer, e.g., from about 1 day to about 14 days, from about 2 days to about 14 days, from about 5 days to about 14 days, from about 7 days to about 14 days, from about 5 days to about 10 days, from about 5 days to about 7 days, or from about 7 days to about 10 days). The second culture period may be from about 1 day to about 14 days (e.g., from about 3 days to about 14 days, from about 3 days to about 12 days, from about 4 days to about 1 days, from about 5 days to about 10 days, or from about 5 days to about 7 days).

In some embodiments, the viral vector is cultured with the primed γδ T cells at a multiplicity of infection (MOI) of no greater than about 10, e.g., no greater than about 9, 8, 7, 6, 5, 4, 3, 2, 1, 0.5, or 0.25. In some embodiments, viral vector is cultured with the primed γδ T cells at a multiplicity of infection (MOI) of no greater than about 5. In some embodiments, viral vector is cultured with the primed γδ T cells at a multiplicity of infection (MOI) of no greater than about 4. In some embodiments, viral vector is cultured with the primed γδ T cells at a multiplicity of infection (MOI) of no greater than about 3. In some embodiments, viral vector is cultured with the primed γδ T cells at a multiplicity of infection (MOI) of no greater than about 2. In some embodiments, viral vector is cultured with the primed γδ T cells at a multiplicity of infection (MOI) of no greater than about 1. In some embodiments, viral vector is cultured with the primed γδ T cells at a multiplicity of infection (MOI) of no greater than about 0.5. In some embodiments, viral vector is cultured with the primed γδ T cells at a multiplicity of infection (MOI) of no greater than about 0.25. In some embodiments, viral vector is cultured with the primed γδ T cells at a multiplicity of infection (MOI) of from about 0.25 to about 10 (e.g., about 0.5 to about 10, about 1 to about 10, or about 1 to about 5).

In some embodiments, transduction of γδ T cells includes the use of a transduction enhancer to enhance transduction efficiency. Suitable transduction enhancers include, e.g., vectorfusin, spermid, and/or retronectin. The methods may include contacting the γδ T cells with the transduction enhancer during culturing. In some embodiments, the method further includes contact the cells with nevirapine. In some embodiments, transduction of γδ T cells includes supplementing the culture medium with a IL-15, which can increase γδ T cell expression of ASCT-2, the viral entry receptor for a betaretroviral pseudotyped viral vector.

### Spinoculation

In some embodiments of the disclosure, γδ T cells may be spun e.g., by centrifugation, while being cultured with a viral vector (e.g., in combination with one or more additional agents described herein). This “spinoculation” process may occur with a centripetal force of, e.g., from about 200×g to about 2,000×g. The centripetal force may be, e.g., from about 300×g to about 1,200×g (e.g., about 300×g, 400×g, 500×g, 600×g, 700×g, 800×g, 900×g, 1,000×g, 1,100×g, or 1,200×g, or more). In some embodiments, the γδ T cells are spun for from about 10 minutes to about 3 hours (e.g., about 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 10⁵ minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or more). In some embodiments, the γδ T cells are spun at room temperature, such as at a temperature of about 25° C.

Exemplary transduction protocols involving a spinoculation step are described, e.g., in Millington et al., PLoS One 4:e6461, 2009; Guo et al., Journal of Virology 85:9824-9833, 2011; O'Doherty et al., Journal of Virology 74:10074-10080, 2000; and Federico et al., Lentiviral Vectors and Exosomes as Gene and Protein Delivery Tools, Methods in Molecular Biology 1448, Chapter 4, 2016, the disclosures of each of which are incorporated herein by reference.

### Viral Vectors

The compositions and methods described herein include the use of betaretroviral pseudotyped viral vectors for efficient transduction of γδ T cells. Viral genomes provide a rich source of vectors that can be used for the efficient delivery of exogenous genes into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors that can be betaretroviral pseudotyped include retrovirus (e.g., Retroviridae family viral vector). Examples of retroviruses are: avian leukosis-sarcoma, avian C-type viruses, mammalian C-type, B-type viruses, D-type viruses, oncoretroviruses, HTLV-BLV group, lentivirus, alpharetrovirus, betaretrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses (MLVs), murine sarcoma viruses, mouse mammary tumor virus, bovine leukemia virus, feline leukemia virus, feline sarcoma virus, avian leukemia virus, human T-cell leukemia virus, baboon endogenous virus (BaEV), Gibbon ape leukemia virus, Mason Pfizer monkey virus, simian immunodeficiency virus, simian sarcoma virus, Rous sarcoma virus, and lentiviruses. Other examples of vectors that can be pseudotyped with betraretrovirus for the present methods are described, for example, in McVey et al., (U.S. Pat. No. 5,801,030), the teachings of which are incorporated herein by reference.

### Retroviral Vectors

In some instances, the viral vector used in the methods and compositions described herein is a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125, 2004, the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope protein, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3-splice site (SA), elongation factor (EF) 1-alpha promoter and 3-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963, 2004, may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3-splice site (SA), elongation factor (EF) 1-alpha promoter and 3-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811, 2001, Osborn et al., Molecular Therapy 12:569, 2005, Szymczak and Vignali, Expert Opin Biol Ther. 5:627, 2005, and Szymczak et al., Nat Biotechnol. 22:589, 2004, the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

Other retroviral vectors (e.g., retroviral backbones) that may be used in conjunction with the compositions and methods described herein include gammaretroviral vectors. Exemplary gamma retroviral vectors are, or are derived from, chick syncytial virus, feline leukemia virus, finkel-biskis-jinkins murine sarcoma virus, gardner-arnstein feline sarcoma virus, gibbon ape leukemia virus, guinea pig type-c oncovirus, hardy-zuckerman feline sarcoma virus, harvey murine sarcoma virus, kirsten murine sarcoma virus, koala retrovirus, moloney murine sarcoma virus, murine leukemia virus, porcine type-c oncovirus, reticuloendotheliosis virus, snyder-theilen feline sarcoma virus, trager duck spleen necrosis virus, viper retrovirus, and woolly monkey sarcoma virus.

In certain embodiments, the viral vector backbone is derived from a lentivirus (LV). In certain embodiments, the viral vector backbone is derived from a third-generation self-inactivation (SIN) lentiviral vector (LV) (e.g., HIV, SIV, or EIAV). In certain embodiments, the viral vector backbone is derived from a LV (e.g.) that is not self-inactivating.

Other retroviral vectors (e.g., retroviral backbones) that may be used in conjuction with the compositions and methods described herein include alpharetroviral vectors. Exemplary alpharetroviral vectors are, or are derived from, avian carcinoma mill hill virus 2, avian leukosis virus, avian myeloblastosis virus, avian myelocytomatosis virus 29, avian sarcoma virus ct10, fujinami sarcoma virus, rous sarcoma virus, ur2 sarcoma virus, and y73 sarcoma virus.

### Betaretoviral Pseuodotype

The viral vectors used in conjunction with the compositions and methods described herein includes a betaretroviral psueodotyped envelope gene. The betaretroviral envelope gene may be from a canonical type B or type D betaretrovirus. The betaretoviral psueodtype may be derived from any suitable betaretrovirus. Betaretroviruses include, for example, mouse mammary tumor virus (MMTV), enzootic nasal tumor virus types 1 and 2 (ENT-1 and ENT-2), siminan retrovirus types 1, 2 (SRV-1 and SRV-2), and 3, jaagsiekte sheep retrovirus (JSRV), squirrel monkey retrovirus (SMRV), Trichosurus Vulpecula endogenous type D retrovirus (TvERV-D), Mus musculus type D retovirus (MusD), simian endogenous retrovirus (SERV), Mason-Pfizer monkey virus MPMV. In some embodiments, the betaretroviral envelope gene is from a non-betaretroviral vector. These viruses have potentially acquired the betaretroviral pseudotype through recombination and cross-species transmission. Suitable examples include BaEV, feline retrovirus RD114, sin nombre virus (SNV), and reticuloendotheliosis virus (REV). Envelope genes that may be used in conjunction with the compositions and methods described herein include those from viruses described in Baillie et al., J. Virol. 78: 5784-5798, 2004, the disclosure of which is hereby incorporated by reference in its entirety.

### γδ T Cells

Gamma delta T cells (γδ T cells) represent a subset of T cells that express on their surface a distinct, defining γδ T-cell receptor (TCR). This TCR is made up of one gamma (γ) and one delta (δ) chain. Human γδ T cells can be broadly classified as one or two types-peripheral blood-resident γδ T cells and non-haematopoietic tissue-resident γδ T cells. Most blood-resident γδ T cells express a Vδ2 TCR, whereas this is less common among tissue-resident γδ T cells, which more frequently use Vδ1 and/or other Vδchains. The invention provides γδ T cells that are transduced with a viral vector encoding a desired transgene as described herein.

In some embodiments, suitable γδ T cells for use as a source for the presently described engineered γδ T cells include Vδ1 cells, Vδ2 cells, Vδ3 cells, Vδ5 cells, and Vδ8 cells. In some embodiments, the population of engineered γδ T cells is derived from a population of Vδ1 cells or Vδ2 cells. In some instances, the population of engineered γδ T cells is derived from a population of non-Vδ1/Vδ2 T cells. In some instances, population of engineered γδ T cells is derived from a mixed population of Vδ1 cells and Vδ2 cells.

The γδ T cells described herein (e.g., endogenous γδ T cells or primed γδ T cells) may lack a vesicular stomatis virus G glycoprotein (VSV-G) entry receptor (e.g., LDL). The γδ T cell (e.g., endogenous γδ T cells or primed γδ T cells) may express ASCT-1 and/or ASCT-2. The expression of ASCT-1 and/or ASCT-2 may permit transduction with a betaretroviral pseudotyped vector (e.g., BaEV and RD114). The lack of expression of VSV-G may prevent transduction with a VSV-G psuedotyped vector.

In one aspect, the invention provides a population of γδ T cells engineered to express one or more transgenes, which may encode a membrane-bound protein (e.g., a cell surface receptor, such as a chimeric antigen receptor (CAR), an as TCR, a natural cytotoxicity receptor (e.g., NKp30, NKp44, or NKp46), a cytokine receptor (e.g., IL-12 receptor), a chemokine receptor (e.g., CCR2 receptor), and/or a membrane-bound ligand or cytokine (e.g., membrane-bound IL-15, membrane-bound IL-7, membrane-bound CD40L, membrane-bound 4-1BB, membrane-bound 4-1BBL, membrane bound CCL19), a soluble protein (e.g., soluble ligands or cytokines, e.g., soluble IL-15, soluble IL-7, soluble IL-12, soluble CD40L, soluble 4-1BBL, and/or soluble CCL19), a selectable marker (e.g., a reporter gene), or a suicide gene. In some instances, the invention provides a population of γδ T cells engineered to express a CAR and one or more additional transgene-encoded proteins (e.g., an armor protein). In some embodiments, the one or more transgenes are codon optimized.

In some embodiments, the γδ T cell is transduced with a viral vector encoding a transgene. In some embodiments, the viral vector is a retroviral vector. In some embodiments, the viral vector is a lentiviral vector. In some such embodiments, the cell may stably express the transgene. In some embodiments, the cell may transiently express the transgene.

In one aspect, the invention features a cell population (e.g., an isolated cell population) of engineered γδ T cells (e.g., at least 10, 10², 10³, 10⁴, 10⁵, 10⁶, 10⁷, 10⁸, 10⁹, 10¹⁰, 10¹¹, 10¹², or 10¹³ cells), wherein at least 3% (e.g., at least 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the cell population are of engineered γδ T cells expressing the transgene (e.g., the CAR and/or one or more additional proteins).

### Methods of Harvesting and Expanding γδ T Cells

Engineered γδ T cells of the invention can be derived from any suitable autologous or allogeneic γδ T cell or population thereof. In some embodiments, suitable γδ T cells for use as a source for the presently described engineered γδ T cells include Vδ1 cells, Vδ2 cells, Vδ3 cells, Vδ5 cells, and Vδ8 cells. In some embodiments, the population of engineered γδ T cell is derived from a population of Vδ1 cells or Vδ2 cells.

For example, provided herein are methods for separating and expanding Vδ1 cells from a non-haematopoietic tissue, such as skin or gut. In other embodiments, suitable γδ T cells can be derived from blood (e.g., peripheral blood). Methods of isolating and expanding Vδ1 cells from blood include those described, for example, in U.S. Pat. No. 9,499,788 and International Patent Publication No. WO 2016/198480, each of which is incorporated herein by reference in its entirety. In some embodiments, suitable γδ T cells can be derived from tumor tissue (e.g., tumor-infiltrating γδ T cells). Alternatively, suitable γδ T cells that can be engineered to express a transgene can be derived from non-haematopoietic tissue according to methods described below.

Isolation and Expansion of γδ T Cells from Blood

In some embodiments, the engineered γδ T cells of the present invention are derived from blood (e.g., peripheral blood) of a subject. For example, engineered γδ T cells may be derived from blood-derived Vδ2 cells or blood-derived Vδ1 cells.

In some embodiments, peripheral blood mononuclear cells (PBMCs) can be obtained from a subject according to any suitable method known in the art. PBMCs can be cultured in the presence of aminobisphosphonates (e.g., zoledronic acid), synthetic phosphoantigens (e.g., bromohydrin pyrophosphate; BrHPP), 2M3B1PP, or 2-methyl-3-butenyl-1-pyrophosphate in the presence of IL-2 for one-to-two weeks to generate an enriched population of Vδ2 cells. Alternatively, immobilized anti-TCRγδ (e.g., pan TCRγδ) can induce preferential expansion of Vδ2 cells from a population of PBMCs in the presence of IL-2, e.g., for approximately 14 days. In some embodiments, preferential expansion of Vδ2 cells from PBMCs can be achieved upon culture of immobilized anti-CD3 antibodies (e.g., OKT3) in the presence of IL-2 and IL-4. In some embodiments, the aforementioned culture is maintained for about seven days prior to subculture in soluble anti-CD3, IL-2, and IL-4. Alternatively, artificial antigen presenting cells can be used to promote preferential expansion of γδ T cells, such as Vδ2 cells. For example, PBMC-derived γδ T cells cultured in the presence of irradiated aAPC, IL-2, and/or IL-21 can expand to generate a population of γδ T cells including a high proportion of Vδ2 cells, moderate proportion of Vδ1 cells, and some double negative cells. In some embodiments of the aforementioned methods, PBMCs can be pre-enriched or post-enriched (e.g., through positive selection with TCRγδ-specific agents or negative selection of TCRap-specific agents). Such methods and other suitable methods for expansion of γδ T cells, such as Vδ2 cells, are described in detail by Deniger et al., Frontiers in Immunology 5, 636: 1-10, 2014, which is incorporated herein by reference in its entirety.

In some embodiments, Vδ1 T cells can be engineered to express a transgene (e.g., a heterologous targeting construct). Any suitable method of obtaining a population of Vδ1 T cells can be used. For example, Almeida et al. (Clinical Cancer Research, 22, 23; 5795-5805, 2016), incorporated herein by reference in its entirety, provides suitable methods of obtaining a population of Vδ1 T cells that can be engineered to express a heterologous targeting construct described herein. For example, in some embodiments, PBMCs are pre-enriched using magnetic bead sorting, which can yield greater than 90% γδ T cells. These cells can be cultured in the presence of one or more factors (e.g., TCR agonists, co-receptor agonists, and/or cytokines, e.g., IL-4, IL-15, and/or IFN-γ) in gas-permeable bioreactor bags for up to 21 days or more. Variations of this method, and other methods of obtaining Vδ1 T cells are suitable as part of the present invention. For example, blood derived Vδ1 T cells can alternatively be obtained using methods described, for example, in U.S. Pat. No. 9,499,788 and International Patent Publication No. WO 2016/198480, each of which is incorporated herein by reference in its entirety.

Separation and Expansion of Non-Haematopoietic Tissue-Resident γδ T Cells from Non-Haematopoietic Tissue

Non-haematopoietic tissue-resident γδ T cells obtained as described below can be suitable vehicles for transgenes described herein, as they can exhibit good tumor penetration and retention capabilities. More detailed methods for isolation and expansion of non-haematopoietic tissue-resident γδ T cells can be found, for example, in PCT Pub. Nos. WO 2020/095058, WO 2020/095059, WO 2017/072367, and GB App. No. 2006989.4, each of which is incorporated herein by reference in its entirety.

Non-haematopoietic tissue-resident γδ T cells (e.g., skin-derived γδ T cells and/or non-Vδ2 T cells, e.g., Vδ1 T cells and/or DN T cells) can be isolated from any human or non-human animal non-haematopoietic tissue that can be removed from a patient to obtain cells suitable for engineering according to the methods of the present invention. In some embodiments, the non-haematopoietic tissue from which the γδ T cells are derived and expanded is skin (e.g., human skin), which can be obtained by methods known in the art. In some embodiments, the skin is obtained by punch biopsy. Alternatively, the methods of isolation and expansion of γδ T cells provided herein can be applied to the gastrointestinal tract (e.g., colon), mammary gland, lung, prostate, liver, spleen, and pancreas. The γδ T cells may also be resident in human cancer tissues, e.g., tumors of the breast or prostate. In some embodiments, the γδ T cells may be from human cancer tissues (e.g., solid tumor tissues). In other embodiments, the γδ T cells may be from non-haematopoietic tissue other than human cancer tissue (e.g., a tissue without a substantial number of tumor cells). For example, the γδ T cells may be from a region of skin (e.g., healthy skin) separate from a nearby or adjacent cancer tissue.

The γδ T cells that are dominant in the blood are primarily Vδ2 T cells, while the γδ T cells that are dominant in the non-haematopoietic tissues are primarily Vδ1 T cells, such that Vδ1 T cells include about 70-80% of the non-haematopoietic tissue-resident γδ T cell population. However, some Vδ2 T cells are also found in non-haematopoietic tissues, e.g., in the gut, where they can include about 10-20% of γδ T cells. Some γδ T cells that are resident in non-haematopoietic tissues express neither Vδ1 nor Vδ2 TCR and we have named them double negative (DN) γδ T cells. These DN γδ T cells are likely to be mostly Vδ3-expressing with a minority of Vδ5-expressing T cells. Therefore, the γδ T cells that are ordinarily resident in non-haematopoietic tissues and that are expanded by the method of the invention are preferably non-Vδ2 T cells, e.g., Vδ1 T cells, with the inclusion of a smaller amount of DN γδ T cells.

In some embodiments, a critical step is the deliberate separation, e.g., after some days or weeks of culture, of non-haematopoietic tissue-resident T cells (e.g., within a mixed lymphocyte population, which may for example include as cells, natural killer (NK) cells, B cells, and γδ2 and non-γδ2 T cells) away from the non-haematopoietic cells (e.g., stromal cells, particularly fibroblasts) of the tissue from which the T cells were obtained. This permits the preferential and rapid expansion over the following days and weeks of non-haematopoietic tissue-derived Vδ1 T cells and DN γδ T cells.

In general, non-haematopoietic tissue-resident γδ T cells are capable of spontaneously expanding upon removal of physical contact with stromal cells (e.g., skin fibroblasts). Thus, the scaffold-based culture methods described above can be used to induce such separation, resulting in de-repression of the γδ T cells to trigger expansion. Accordingly, in some embodiments, no substantial TCR pathway activation is present during the expansion step (e.g., no exogenous TCR pathway activators are included in the culture). Further, the invention provides methods of expanding non-haematopoietic tissue-resident γδ T cells, wherein the methods do not involve contact with feeder cells, tumor cells, and/or antigen-presenting cells.

Expansion protocols involve culturing non-haematopoietic tissue-resident γδ T cells in the presence of effective cocktails of biological factors to support efficient γδ T cell expansion. In one embodiment, the method of expanding γδ T cells includes providing a population of γδ T cells obtained from a non-haematopoietic tissue (e.g., a separated population of non-haematopoietic tissue-derived γδ T cells, e.g., a population separated according to the methods described herein) and culturing the γδ T cells in the presence of IL-2 and, IL-15, and optionally IL-1β , IL-4, and/or IL-21. These cytokines or analogues thereof can be cultured with the cells for a duration (e.g., at least 5 days, at least 6 days, at least 7 days, at least 8 days, at least 9 days, at least 10 days, at least 11 days, at least 12 days, at least 13 days, at least 14 days, at least 21 days, at least 28 days, or longer, e.g., from 5 days to 40 days, from 7 days to 35 days, from 14 days 28 days, or about 21 days) in an amount effective to produce an expanded population of γδ T cells.

Numerous basal culture media suitable for use in the priming and/or expansion of γδ T cells are available, such as complete media, OPTMIZER™, AIM-V, Iscoves medium and RPMI-1640 (Life Technologies) and TEXMACS™ (Miltenyi Biotec). The medium may be supplemented with other media factors, such as serum, serum proteins and selective agents, such as antibiotics. For example, in some embodiments, a media includes RPMI-1640 containing 2 mM glutamine, 10% FBS, 10 mM HEPES, pH 7.2, 1% penicillin-streptomycin, sodium pyruvate (1 mM; Life Technologies), non-essential amino acids (e.g., 100 μM Gly, Ala, Asn, Asp, Glu, Pro and Ser; 1X MEM non-essential amino acids Life Technologies), and 10 μl/L β-mercaptoethanol. Conveniently, cells are cultured at 37° C. in a humidified atmosphere containing 5% CO₂ in a suitable culture medium.

The γδ T cells may be cultured as described herein in any suitable system, including stirred tank fermenters, airlift fermenters, roller bottles, culture bags or dishes, and other bioreactors, such as hollow fiber bioreactors. The use of such systems is well-known in the art. General methods and techniques for culture of lymphocytes are well-known in the art.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected.

### Transgenes

The engineered γδ T cells of the present invention are engineered to express a desired transgene. γδ T cells engineered to express a transgene are suitable for use in cancer treatment (e.g., immunotherapy). The viral vectors described herein encode the transgene, which is then stably or transiently expressed in the transduced γδ T cells. Transgenes that can be used in conjunction with the compositions and methods described herein include chimeric antigen receptors (CARs).

In some embodiments, the CAR targets CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gpl20, HIV-1 envelope glycoprotein gp41, GD2, CD123, CD33, CD138, CD23, CD30, CD56, c-Met, mesothelin, GD3, HERV-K, IL-IIRalpha, kappa chain, lambda chain, CSPG4, ERBB2, EGFRvIII, VEGFR2, HER2-HER3 in combination, HER1-HER2 in combination, NY-ESO-1, synovial sarcoma X breakpoint 2 (SSX2), melanoma antigen (MAGE), melanoma antigen recognized by T cells 1 (MART-1), gp100, prostate specific antigen (PSA), prostate specific membrane antigen (PSMA), prostate stem cell antigen (PSCA), g9d2, or a combination thereof.

In some instances, a transgene to be expressed by the engineered γδ T cells of the present invention includes a selectable marker (e.g., a reporter gene) or a suicide gene. For example, truncated epidermal growth factor receptor (EGFR), lacking the intracellular signaling domain, can be used as a transgene for in vivo depletion in the event of, e.g., toxicity, using anti-EGFR monoclonal antibodies. Similarly, CD20 can be used as a transgene for in vivo depletion using anti-CD20 monoclonal antibodies. Another exemplary transgene is a suicide gene to facilitate drug-mediated control of administered engineered γδ T cells. Through the use of a suicide gene, modified cells can be depleted from the patient in case of an adverse event. In one example, a drug-binding domain is fused to the caspase9 pro-apoptotic molecule. In some instances, the transgene is cytosine deaminase. In some instances, the transgene is thymidine kinase.

Additionally or alternatively, transgenes for expression by the engineered γδ T cells of the present invention encode membrane-bound proteins, such as a membrane-bound receptor (e.g., asP TCR, a natural cytotoxicity receptor (e.g., NKp30, NKp44, or NKp46), a cytokine receptor (e.g., IL-12 receptor), and/or a chemokine receptor (e.g., CCR2 receptor) and/or a membrane-bound ligand or cytokine (e.g., membrane-bound IL-15, membrane-bound IL-7, membrane-bound CD40L, membrane-bound 4-1BB, membrane-bound 4-1BBL, membrane bound CCL19). Membrane-bound ligands and cytokines include naturally membrane-bound ligands and cytokines (e.g., trans-presented IL-15 and 4-1 BBL) and synthetic membrane-bound configurations (e.g., ligands that have been artificially fused to a trans-membrane protein). Additionally, or alternatively, transgenes to be expressed by the engineered γδ T cells of the present invention encode soluble proteins, such as soluble ligands or cytokines (e.g., soluble IL-15, soluble IL-7, soluble IL-12, soluble CD40L, soluble 4-1BBL, and/or soluble CCL19).

In some instances, engineered γδ T cells having a transgene that encodes a CAR can be armored with an additional transgene that contributes to immunogenicity. Such armored CAR T cells express an armor protein, such as any of the membrane-bound or soluble proteins described herein. For example, armor proteins include membrane-bound proteins, such as a membrane-bound receptor (e.g., asP TCR, a natural cytotoxicity receptor (e.g., NKp30, NKp44, or NKp46), a cytokine receptor (e.g., IL-12 receptor), and/or a chemokine receptor (e.g., CCR2 receptor) and/or a membrane-bound ligand or cytokine (e.g., membrane-bound IL-15, membrane-bound IL-7, membrane-bound CD40L, membrane-bound 4-1BB, membrane-bound 4-1BBL, membrane bound CCL19). Additionally, or alternatively, armor proteins to be expressed by the engineered γδ CAR T cells of the present invention include soluble proteins, such as soluble ligands or cytokines (e.g., soluble IL-15, soluble IL-7, soluble IL-12, soluble CD40L, soluble 4-1BBL, and/or soluble CCL19).

In some embodiments, the engineered γδ T cells of the present invention are engineered to express one or more transgenes (e.g., one or more of any of the transgenes described herein) for armoring the γδ T cells (e.g., as an armored CAR T cell, as described in Yeku and Brentjens Biochem. Soc. Trans. 2016, 15: 44, 2, 412-418, which is incorporated herein by reference in its entirety).

In some embodiments, the transgene is codon-optimized.

In some embodiments, at least 3% (e.g., at least 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the engineered population of γδ T cells (e.g., Vδ1 or Vδ2 cells) express the transgene, e.g., the CAR or other membrane-bound or soluble protein. In some embodiments, at least 10% (e.g., at least 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99%, or substantially all) of the engineered population of γδ T cells (e.g., Vδ1 or Vδ2 cells) express the transgene, e.g., the CAR or other membrane-bound or soluble protein. In some embodiments, at least 50% (e.g., at least 50%, 55%, 60%, 65%, 70%, 75%, 80%, 85%, 90%, 95%, 97%, 99%, or substantially all) of the engineered population of γδ T cells (e.g., Vδ1 or Vδ2 cells) express the transgene, e.g., the CAR or other membrane-bound or soluble protein. In some embodiments, 3%-95% (e.g., 5%-95%, 10%-95%, 20%-95%, 25%-95%, or 50%-95%) of the engineered population of γδ T cells (e.g., Vδ1 or Vδ2 cells) express the transgene, e.g., the CAR or other membrane-bound or soluble protein. In some embodiments, 3%-90% (e.g., 5%-90%, 10%-90%, 20%-90%, 25%-90%, or 50%-90%) of the engineered population of γδ T cells (e.g., Vδ1 or Vδ2 cells) express the transgene, e.g., the CAR or other membrane-bound or soluble protein.

### EXAMPLES

### Materials and Methods

### Retroviral Vector Production and Titration

Lentiviral vectors were produced by transient transfection of HEK293 cells with a third-generation self-inactivating vector platform consisting of genome (GFP or anti-CD19 chimeric antigen receptor), gag/pol, reverse transcriptase (rev) and envelope (VSV-G, BaEV) encoding plasmids.

Gammaretroviral vectors were produced by transient transfection of FLYRD18 cells with murine leukaemia virus genome plasmid (GFP or anti-CD19 chimeric antigen receptor). Vectors were harvested 48 hours post-transfection, filtered through 0.45 um pore size polyethersulfone (PES) filters and concentrated using low-speed centrifugation (6,000 g at 4° C.).

Vector titre was determined by transduction of human cervical carcinoma cell line (HeLa) with serial dilution of concentrated vector material in the presence of polybrene (8 ug/mL). Transduction efficiency was determined three days post transduction using a BD FACS Lyric flow cytometer.

Infectious titre (TU/mL) was calculated using the following formula: TU/mL=((number of transduced cells)×vector dilution×(% transduction efficiency/100))/volume of vector (mL).

### Flow Cytometry

Immunophenotyping was performed using a BD FACS Lyric flow cytometer. Cells were analysed for the expression of surface markers using PerCP-Vio700 anti-TCR α/β (Miltenyi), APC anti-TCR γ/δ (Miltenyi) and VioBlue anti-TCR Vδ1 (Miltenyi) antibodies. Viable cells were detected using eFluor 780 fixable viability dye. CAR19 expression was detected using FITC labelled human CD19 protein (AcroBiosystems).

### γδ T-Cell Isolation and Expansion

Vδ1 γδ T-cell enriched product (GDX012) was produced using a modified protocol based on Almeida et al. Clin. Cancer Res. 22: 5795-804, 2016. Briefly, αβ-depleted peripheral blood mononuclear cells were expanded using serum-free culture medium (CTS OpTmizer, Thermo Fisher) supplemented with 2.5% autologous plasma and Glutamax (ThermoFisher). The isolated cells were grown in the presence of recombinant IL-4 [rIL4](100 ng/mL), recombinant interferon-γ [rIFNγ](70 ng/mL), recombinant IL-21 [rIL21](7 ng/mL), recombinant IL-1β [rIL1β](15 ng/mL, and soluble OKT-3 anti-CD3 monoclonal antibody (70 ng/mL). Cells were incubated at 37° C. and 5% CO2 in a humidified incubattor. Expanding cells were regularly fed with fresh medium containing recombinant IL-15 [rIL15](70 ng/mL), IFNγ (30 ng/mL), and OKT3 (1 mg/mL).

### Retroviral Transduction

Expanding γδ T-cells were transduced with retroviral vectors at defined multiplicity of infection (MOI). MOI refers to the number of infectious particles (measured by flow cytometry) that were added per cell during transduction. γδ T cells (1 E+06/mL) were transduced in RetroNectin coated (20 g/mL) non-tissue culture treated 24-well plates or in 24 well plates in the presence of vectofusin (1 g/mL). Viral vector was diluted in CTS OpTmizer medium supplemented with cytokines, OKT-3 and 2.5% autologous plasma (as above). γδ T cells and vector stocks were spinoculated at 1,000×g for 2 hours at 37° C. Transduction efficiency was determined using flow cytometry after three days post-transduction at regular intervals. In certain experiments, to inhibit reverse transcriptase activity, the culture media was supplemented with nevirapine (NVP), a non-nucleoside reverse transcriptase inhibitor, at 10 μM final concentration.

### Example 1. Broad Tropism VSV-G Pseudotyped Lentiviral Vectors Cannot Transduce γδ T-Cells

GFP encoding lentiviral vectors were pseudotyped with vesicular stomatitis virus G (VSV-G) or baboon endogenous virus (BaEV) envelope, respectively. Expanded γδ T-cells (consisting of Vδ1, Vδ2 and non-Vδ1/Vδ2 cells) were transduced with concentrated viral vector stocks at defined multiplicity of infections (MOI). Transduction efficiency was determined using flow cytometry three days post-transduction.

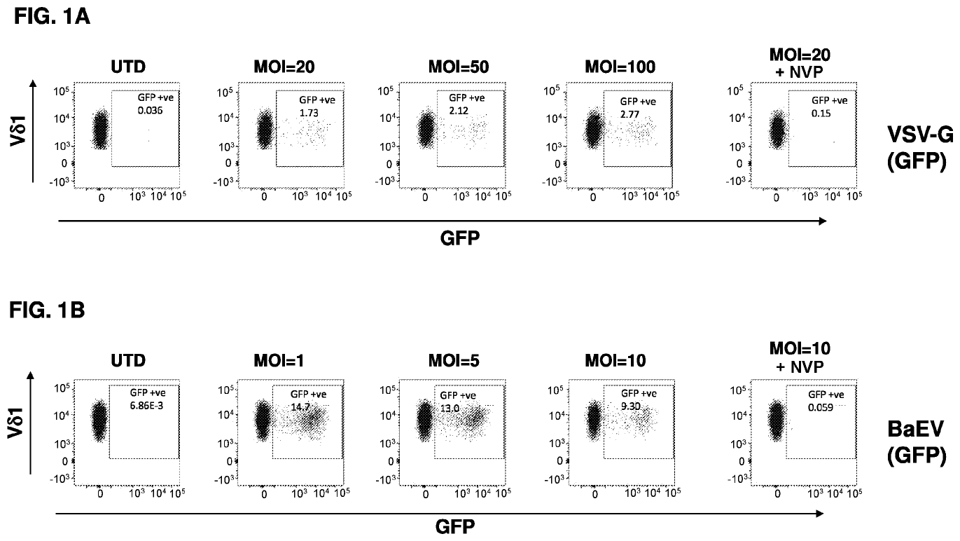

Flow cytometry analysis revealed that VSV-G psdeudotyped lentiviral vectors fail to transduce γδ T cells even at high MOIs (MOI 50 and above, FIG. 1A). On the contrary, transduction with BaEV enveloped lentiviral vector resulted in high transduction efficiencies even at low multiplicity of infections (FIG. 1B). Pre-treatment of γδ T cells with the reverse transcriptase inhibitor NVP abolished GFP expression, indicating that GFP expression was a result of successful transduction and GFP expression in Vδ1 cells.

### Example 2. Transduction of Vδ1 γδ T-Cells with VSV-G Pseudotyped CAR Encoding Lentiviral Vectors Result in Pseudotransduction

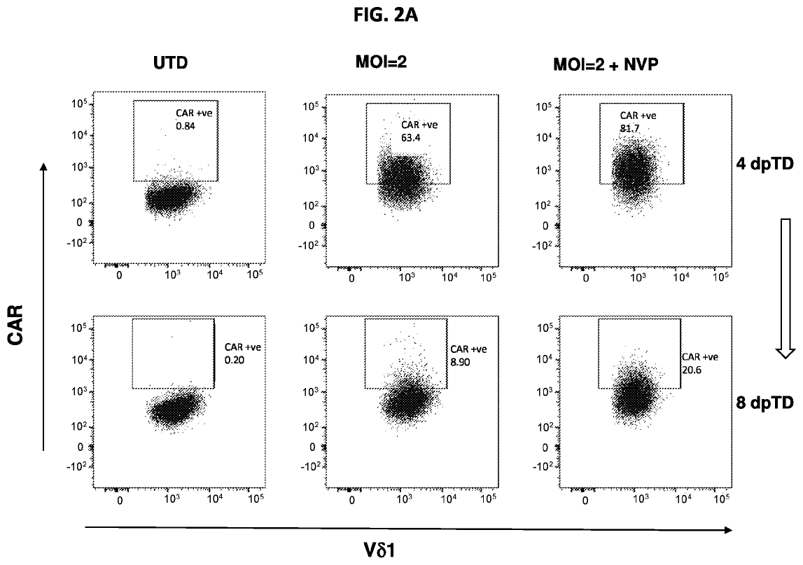

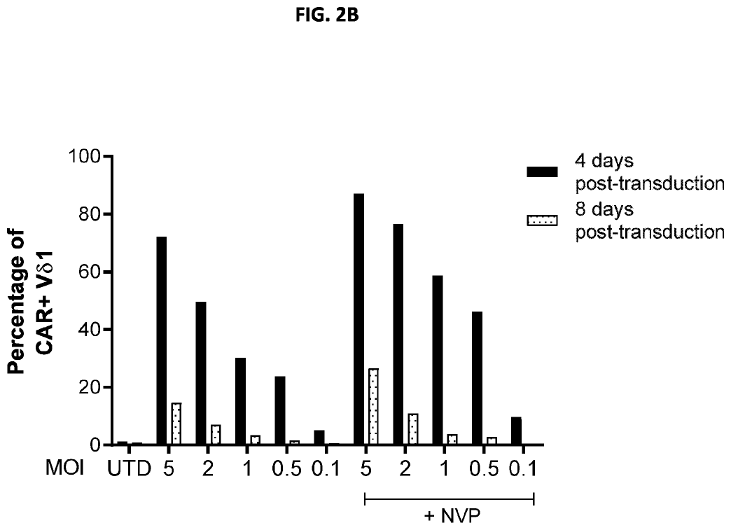

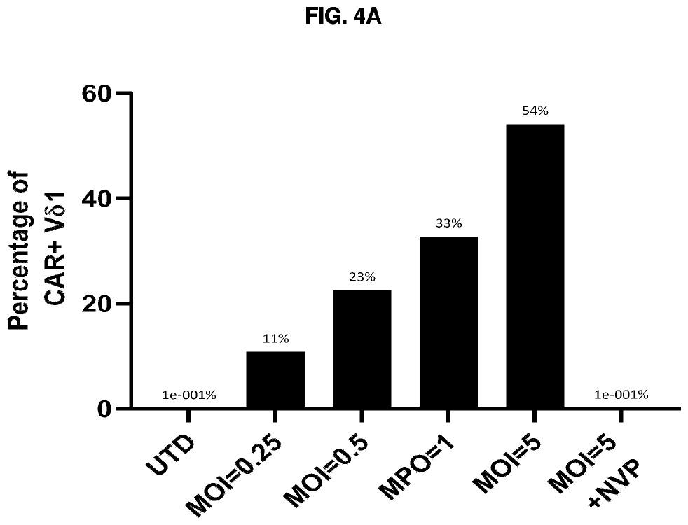

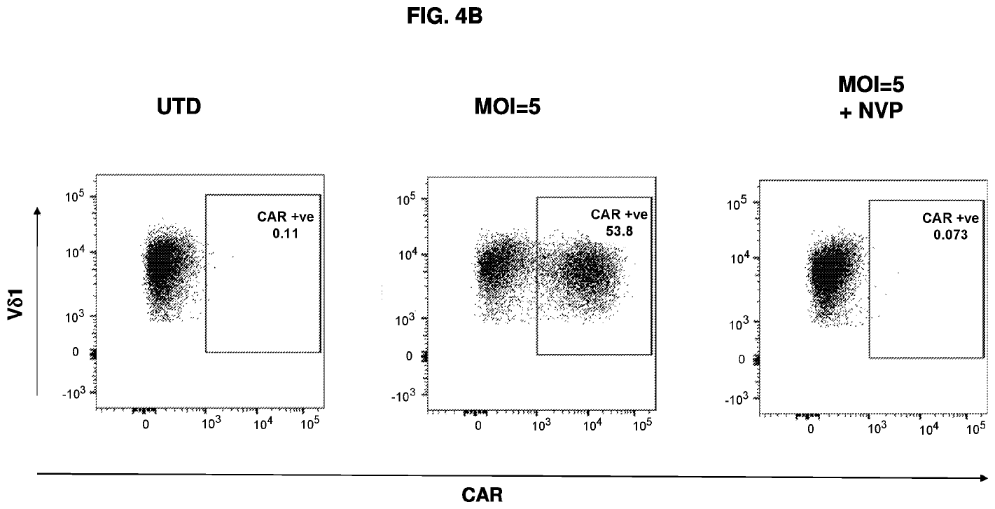

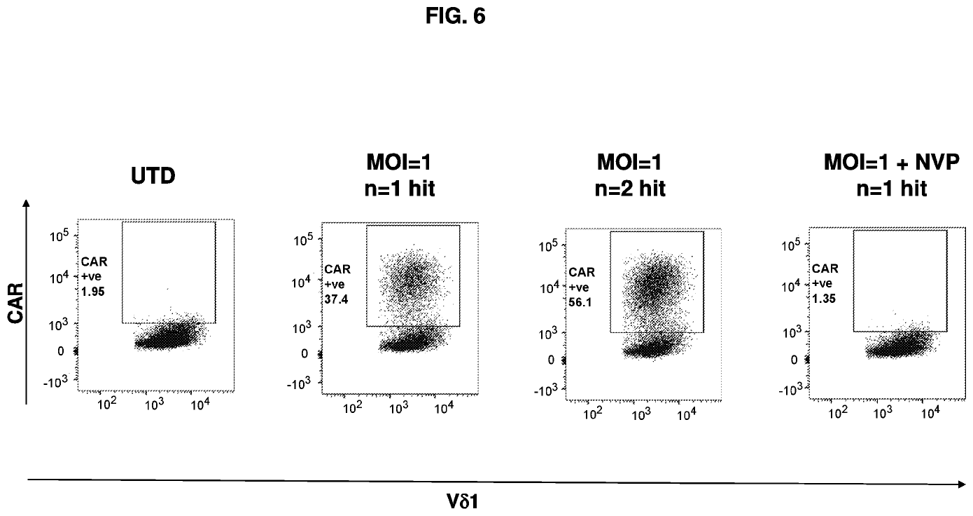

To determine whether CAR expression was a result of vector integration or pseudotransduction, Vδ1 γδ T cells were transduced with chimeric antigen receptor encoding lentiviral vectors in the presence or absence of nevirapine (NVP). Nevirapine is a reverse transcriptase inhibitor that blocks viral transduction by inhibiting the reverse transcription of viral RNA to cDNA. Hence, incubation of the cells exposed to lentiviral vectors in the presence of nevirapine should diminish transgene expression. CAR expression was completely abolished when transduction with BaEV pseudotyped vector was performed in the presence of nevirapine, demonstrating that CAR expression did not result from pseudotransduction (FIGS. 4B and 6). On the contrary, treatment of Vδ1 cells with nevirapine did not abrogate CAR expression in cells transduced with VSV-G pseudotyped lentiviral vector. This result demonstrates that VSV-G pseudotyped vectors are not able to transduce Vδ1 cells, and transgene (CAR) expression is a result of pseudotransduction. Pseudotransduction was further confirmed by monitoring CAR expression over extended periods of time following transductions (4 and 8-days post-transduction). Monitoring the vector treated cells by FACS analysis revealed that the CAR expression was gradually lost over the time (FIG. 2A). This phenomenon was also demonstrated across various multiplicity of infections in the presence or absence NVP (FIG. 2B). Overall, the results suggest that VSV-G pseudotyped lentiviral vectors cannot enter to γδ T cells.

### Example 3. Cytokine Priming is a Major Determinant of Vδ1 γδ T Cells Transduction by BaEV Pseudotyped Lentiviral Vectors

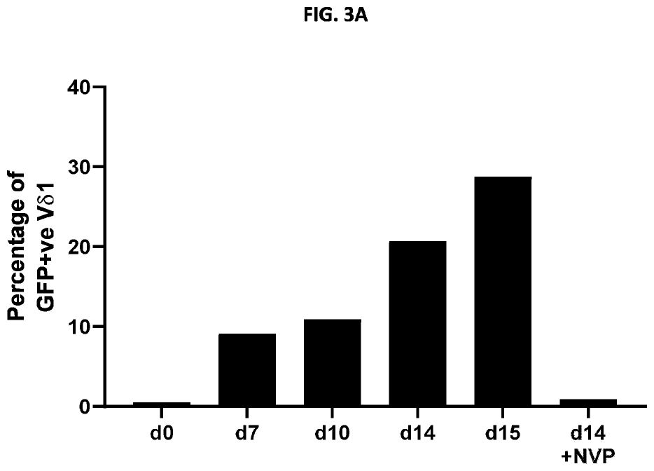

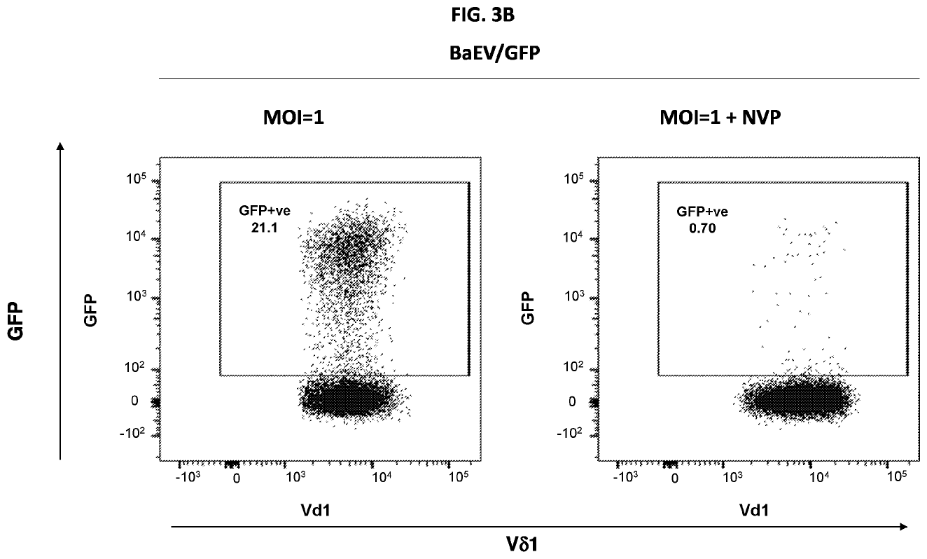

To investigate whether the BaEV transduction efficiency was dependent on the length of cytokine priming during γδ T cell expansion, Vδ1 cells were transduced at different time points during the cell expansion process. Cells were transduced with MOI=1 at the start of the culture (day 0) or at day7, day 10, day 14 and day 15 of the expansion phase. Transduced cells were analysed by flow cytometry for GFP expression three days post-transduction. Transduction efficiencies were gradually increased during the cell expansion phase and reached the highest level of transduction at day 15 (FIG. 3A). Treatment of the cells with NVP demonstrated that the GFP expression was a consequence of successful vector integration (FIG. 3B). Overall, the results suggest that an initial ‘cytokine priming’ phase is necessary for successful Vδ1 transduction by BaEV pseudotyped lentiviral vectors.

### Example 4. Transduction Efficiency of Vδ1 γδ T Cells Correlates with the Multiplicity of Infection (MOI)

To investigate whether the BaEV transduction efficiency was dependent on the viral vector dose (MOI) Vδ1 γδ T cells were transduced with increasing amount of BaEV envelope pseudoyped anti-CD19 chimeric antigen receptor (CAR) encoding lentiviral vectors. Three days post-transduction, cells were analysed by flow cytometry for CAR expression. Increasing the MOI significantly increased the proportion of transduced Vδ1 cells (FIG. 4A). Representative dot plots of CAR transductions at MOI=5 in the presence or absence of NVP are shown in FIG. 4B.

### Example 5. BaEV Pseudotyped Lentiviral Vectors Transduce Both Vδ1 and Non-Vδ1 (Vδ2, Vδ3) γδ T Cells

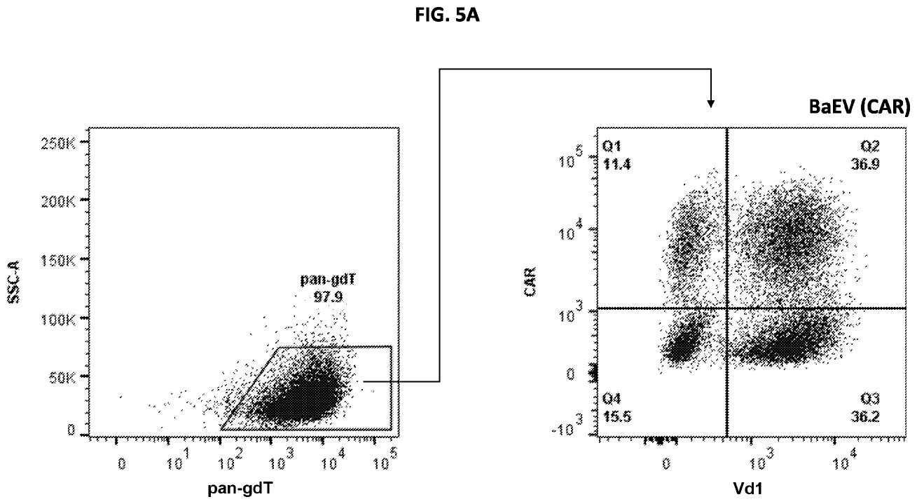

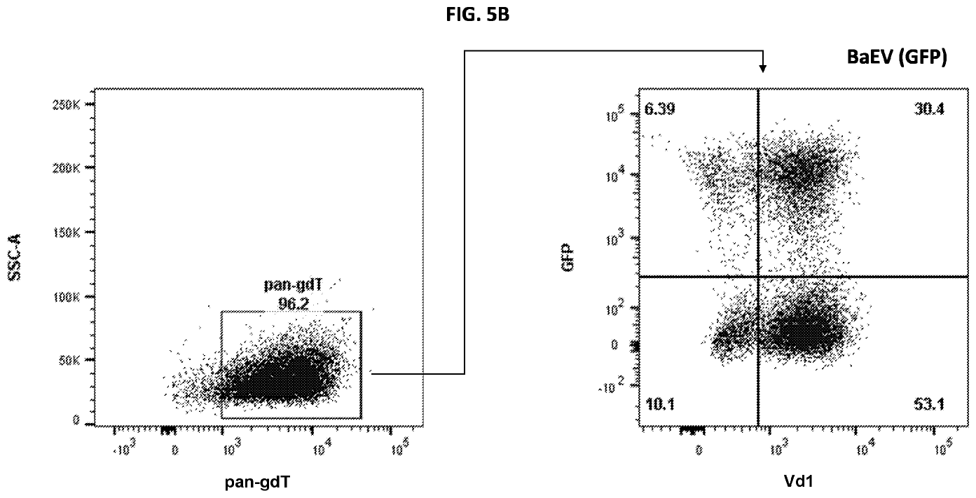

To test if BaEV pseudotyped vectors exclusively transduce Vδ1 cells or can transduce other γδ T cell subtypes as well, transduction efficiencies were determined within the pan-γδ and Vδ1 cell populations. γδ T cells were expanded and transduced on day 10 of the expansion with GFP or CAR encoding BaEV enveloped lentiviral vectors at MOI=1. FACS analysis using pan-γδ and Vδ1 specific antibodies revealed that BaEV enveloped vectors transduced both Vδ1 and non-Vδ1 (Vδ2, Vδ3 and other) γδ T cells (FIG. 5).

### Example 6. Transduction of Vδ1 γδ T Cells with BaEV Pseudotyped Lentiviral Vectors can be Further Enhanced by Repeated Transductions

Studies were undertaken to determine if consecutive transductions could further enhance CAR expression in expanded Vδ1 γδ T cells. To this end Vδ1 cells were transduced with MOI=1 either once (on day 10) or twice (on days 10 and 11). Three days later, the cells were collected and analysed by FACS. Flow cytometry analysis revealed that Vδ1 cells can be efficiently transduced with a single vector hit, an effect which could be further enhanced by double transduction on consecutive days (FIG. 6).

### Example 7. Transduction in the Presence of Vectofusin is as Efficient as in the Presence of Retronectin

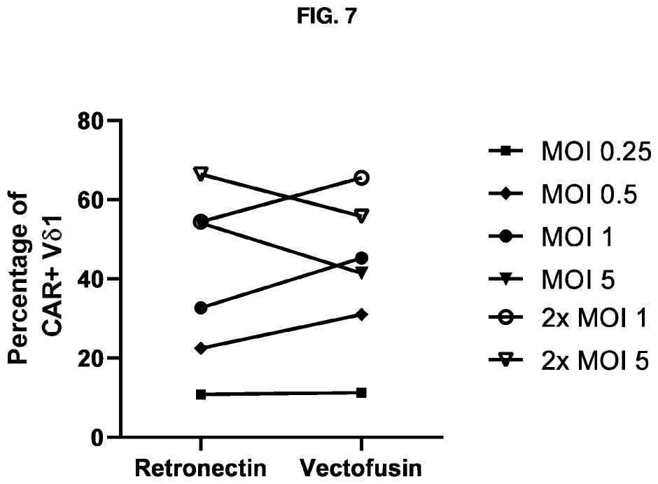

To test whether the choice of transduction enhancer has any influence on Vδ1 transduction efficiencies, two widely used transduction enhancer (retronectin and vectofusin) were evaluated. At day 10 of the cell expansion, Vδ1 cells were transduced with various MOIs in the presence of retronectin or vectofusin and transduction efficiencies were determined three days post-transduction. FACS analysis revealed that vectofusin was as efficient to increase retroviral gene transfer as retronectin (FIG. 7).

### Example 8. Vδ1 Cells can be Transduced with RD114 Pseudotyped Viral Vectors

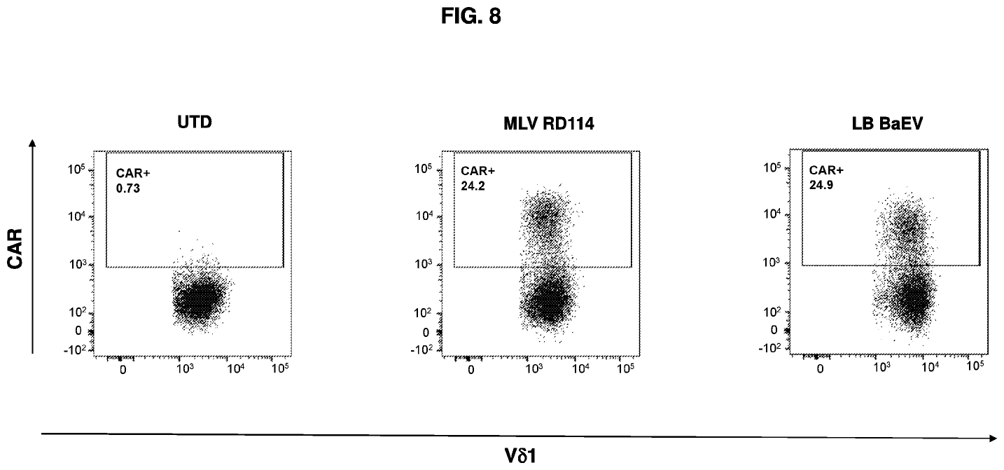

To test whether Vδ1 γδ T cells can be transduced by other betaretroviral viral envelope pseudotyped vectors, Vδ1 γδ T cells were also transduced with RD114 envelope pseudotyped gammaretroviral vectors. Cells were expanded as before and transduced with MOI=1 at day 10 of the expansion. FACS analysis revealed that similarly to BaEV pseudotyped lentiviral vectors, RD114 enveloped gammaretroviral vectors were able to transduce Vδ1 γδ T cells with high efficiency (FIG. 8).

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method of producing a population of engineered γδ T cells, the method comprising transducing a population of γδ T cells with a viral vector comprising a betaretroviral pseudotype and a Retroviridae family viral vector backbone.

2. The method of claim 1, wherein the betaretroviral pseudotype is baboon endogenous virus (BaEV).

3. The method of claim 1, wherein the betaretroviral pseudotype is RD114.

4. The method of any one of claims 1-3, wherein the Retroviridae family viral vector backbone is a retroviral vector backbone.

5. The method of claim 4, wherein the retroviral vector backbone is a lentiviral backbone.

6. The method of claim 4, wherein the retroviral vector backbone is a gammaretroviral backbone.

7. The method of claim 4, wherein the retroviral vector backbone is an alpharetroviral backbone.

8. The method of any one of claims 1-7, wherein the engineered γδ T cells are Vδ1 T cells.

9. The method of any one of claims 1-7, wherein the engineered γδ T cells are Vδ2 T cells.

10. The method of any one of claims 1-7, wherein the engineered γδ T cells are non-Vδ1/Vδ2 T cells.

11. The method of any one of claims 1-10, wherein the viral vector comprises a transgene.

12. The method of claim 11, wherein the transgene encodes a cell surface receptor.

13. The method of claim 12, wherein the cell surface receptor is a chimeric antigen receptor (CAR).

14. The method of claim 13, wherein the CAR targets CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gpl20, HIV-1 envelope glycoprotein gp41, GD2, CD123, CD33, CD138, CD23, CD30, CD56, c-Met, mesothelin, GD3, HERV-K, IL-IIRalpha, kappa chain, lambda chain, CSPG4, ERBB2, EGFRvIII, VEGFR2, HER2-HER3 in combination, HER1-HER2 in combination, NY-ESO-1, synovial sarcoma X breakpoint 2 (SSX2), melanoma antigen (MAGE), melanoma antigen recognized by T cells 1 (MART-1), gp100, prostate specific antigen (PSA), prostate specific membrane antigen (PSMA), prostate stem cell antigen (PSCA), g9d2, or a combination thereof.

15. The method of any one of claims 11-14, wherein the transgene encodes a cytokine.

16. The method of claim 15, wherein the cytokine is secreted.

17. The method of claim 15, wherein the cytokine is membrane-bound.

18. The method of any one of claims 15-17, wherein the cytokine is IL-15.

19. A method of producing a population of engineered γδ T cells, the method comprising:
(i) providing a starting population of γδ T cells;
(ii) culturing the starting population of γδ T cells for a first culture period in the absence of a viral vector to produce a population of primed γδ T cells; and
(iii) culturing the population of primed γδ T cells for a second culture period in the presence of a viral vector comprising a betaretroviral pseudotype in an amount effective to transduce at least 3% of the primed γδ T cells, thereby producing the population of engineered γδ T cells.

20. The method of claim 19, wherein the first culture period is for 1 day or longer.

21. The method of claim 20, wherein the first culture period is for 2 days or longer.

22. The method of any one of claims 19-21, wherein the second culture period is for 2 days or longer.

23. The method of claim 22, wherein the second culture period is for 3 days or longer.

24. The method of any one of claims 19-23, wherein the population of primed γδ T cells expresses ASCT-1 and/or ASCT-2.

25. The method of any one of claims 19-24, wherein the population of primed γδ T cells lacks functional expression of a VSV-G entry receptor.

26. The method of any one of claims 19-25, wherein the viral vector is in an amount effective to transduce at least 20% of the primed γδ T cells.

27. The method of any one of claims 19-26, wherein the viral vector is cultured with the primed γδ T cells at a multiplicity of infection (MOI) no greater than 10.

28. The method of claim 27, wherein the MOI is no greater than 5.

29. The method of claim 28, wherein the MOI is from 1 to 5.

30. A method of producing a population of engineered γδ T cells, the method comprising:
(i) providing a starting population of γδ T cells; and
(ii) culturing the starting population of γδ T cells in the presence of IL-15 and a viral vector comprising a betaretroviral pseudotype in an amount effective to transduce at least 3% of the starting population of γδ T cells, thereby producing the population of engineered γδ T cells.

31. The method of claim 30, wherein the starting population of γδ T cells lack expression of ASCT-1 or ASCT-2.

32. The method of claim 31, wherein the starting population of γδ T cells lack expression of ASCT-1 and ASCT-2.

33. The method of any one of claims 30-32, wherein the starting population of γδ T cells expresses ASCT-1 and/or ASCT-2.

34. The method of any one of claims 30-33, wherein the starting population of γδ T cells lacks expression of a VSV-G entry receptor.

35. The method of claim 34, wherein the VSV-G entry receptor is an LDL receptor.

36. The method of any one of claims 30-35, wherein the viral vector is cultured with the starting population of γδ T cells at an MOI no greater than 10.

37. The method of claim 36, wherein the MOI is from 1 to 10.

38. The method of any one of claims 35-37, wherein the MOI is no greater than 5.

39. The method of claim 38, wherein the MOI is from 1 to 5.

40. The method of any one of claims 19-39, wherein the betaretroviral pseudotype is BaEV.

41. The method of any one of claims 19-39, wherein the betaretroviral pseudotype is RD114.

42. The method of any one of claims 19-41, wherein the viral vector comprises a Retroviridae family viral vector backbone.

43. The method of claim 42, wherein the Retroviridae family viral vector backbone is a retroviral vector backbone.

44. The method of claim 43, wherein the retroviral vector backbone is a lentiviral backbone.

45. The method of claim 43, wherein the retroviral vector backbone is a gammaretroviral backbone.

46. The method of claim 43, wherein the retroviral vector backbone is an alpharetroviral backbone.

47. The method of any one of claims 19-46, wherein the engineered γδ T cells are Vδ1 T cells.

48. The method of any one of claims 19-46, wherein the engineered γδ T cells are Vδ2 T cells.

49. The method of any one of claims 19-46, wherein the engineered γδ T cells are non-Vδ1/Vδ2 T cells.

50. The method of any one of claims 19-49, wherein the viral vector comprises a transgene.

51. The method of claim 50, wherein the transgene encodes a cell surface receptor.

52. The method of claim 51, wherein the cell surface receptor is CAR.

53. The method of claim 52, wherein the CAR targets CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gpl20, HIV-1 envelope glycoprotein gp41, GD2, CD123, CD33, CD138, CD23, CD30, CD56, c-Met, mesothelin, GD3, HERV-K, IL-IIRalpha, kappa chain, lambda chain, CSPG4, ERBB2, EGFRvIII, VEGFR2, HER2-HER3 in combination, HER1-HER2 in combination, NY-ESO-1, SSX2, MAGE, MART-1, gp100, PSA, PSMA, PSCA, g9d2, or a combination thereof.

54. The method of any one of claims 50-53, wherein the transgene encodes a cytokine.

55. The method of claim 54, wherein the cytokine is secreted.

56. The method of claim 55, wherein the cytokine is membrane-bound.

57. The method of any one of claims 54-56, wherein the cytokine is IL-15.

58. A method of producing a population of γδ T cells expressing a CAR, the method comprising transducing a population of γδ T cells with a viral vector comprising:
(i) a transgene encoding the CAR;
(ii) a betaretroviral pseudotype; and
(iii) a Retroviridae family viral vector backbone.

59. A method of producing a population of γδ T cells expressing a CAR and an armor protein, the method comprising transducing a population of γδ T cells with a viral vector comprising:
(i) a first transgene encoding the CAR;
(ii) a second transgene encoding the armor protein;
(iii) a betaretroviral pseudotype; and
(iv) a Retroviridae family viral vector backbone.

60. The method of claim 59, wherein the armor protein is a cytokine.

61. The method of claim 60, wherein the cytokine is secreted.

62. The method of claim 61, wherein the cytokine is membrane-bound.

63. The method of any one of claims 60-62, wherein the cytokine is IL-15.

64. The method of any one of claims 58-63, wherein the betaretroviral pseudotype is BaEV.

65. The method of any one of claims 58-63, wherein the betaretroviral pseudotype is RD114.

66. The method of any one of claims 58-65, wherein the Retroviridae family viral vector backbone is a retroviral vector backbone.

67. The method of claim 66, wherein the retroviral vector backbone is a lentiviral backbone.

68. The method of claim 66, wherein the retroviral vector backbone is a gammaretroviral backbone.

69. The method of claim 66, wherein the retroviral vector backbone is an alpharetroviral backbone.

70. The method of any one of claims 58-69, wherein the γδ T cells are Vδ1 T cells.

71. The method of any one of claims 58-69, wherein the γδ T cells are Vδ2 T cells.

72. The method of any one of claims 58-69, wherein the γδ T cells are non-Vδ1/Vδ2 T cells.

73. A method of producing a population of γδ T cells expressing a CAR, the method comprising:
(i) providing a starting population of γδ T cells;
(ii) culturing the starting population of γδ T cells for a first culture period in the absence of a viral vector to produce a population of primed γδ T cells; and
(iii) culturing the population of primed γδ T cells for a second culture period in the presence of a viral vector comprising a betaretroviral pseudotype and a transgene encoding the CAR, wherein the viral vector is in an amount effective to transduce at least 3% of the primed γδ T cells, thereby producing the population of γδ T cells expressing the CAR.

74. A method of producing a population of γδ T cells expressing a CAR and an armor protein, the method comprising:
(i) providing a starting population of γδ T cells;
(ii) culturing the starting population of γδ T cells for a first culture period in the absence of a viral vector to produce a population of primed γδ T cells; and
(iii) culturing the population of primed γδ T cells for a second culture period in the presence of a viral vector comprising a betaretroviral pseudotype, a first transgene encoding the CAR, and a second transgene encoding the armor protein, wherein the viral vector is in an amount effective to transduce at least 3% of the primed γδ T cells, thereby producing the population of γδ T cells expressing the CAR and the armor protein.

75. The method of claim 74, wherein the armor protein is a cytokine.

76. The method of claim 75, wherein the cytokine is secreted.

77. The method of claim 75, wherein the cytokine is membrane-bound.

78. The method of any one of claims 74-77, wherein the cytokine is IL-15.

79. The method of any one of claims 73-78, wherein the first culture period is for 7 days or longer.

80. The method of claim 79, wherein the first culture period is for 10 days or longer.

81. The method of any one of claims 73-80, wherein the second culture period is for 7 days or longer.

82. The method of claim 81, wherein the second culture period is for 14 days or longer.

83. The method of any one of claims 73-82, wherein the population of primed γδ T cells expresses ASCT-1 and/or ASCT-2.

84. The method of any one of claims 78-83, wherein the population of primed γδ T cells lacks functional expression of a VSV-G entry receptor.

85. The method of any one of claims 73-84, wherein the viral vector is in an amount effective to transduce at least 20% of the primed γδ T cells.

86. The method of any one of claims 73-85, wherein the viral vector is cultured with the primed γδ T cells at an MOI no greater than 10.

87. The method of claim 86, wherein the MOI is no greater than 5.

88. The method of claim 87, wherein the MOI is from 1 to 5.

89. A method of producing a population of γδ T cells expressing a CAR, the method comprising:
(i) providing a starting population of γδ T cells; and
(ii) culturing the starting population of γδ T cells in the presence of IL-15 and a viral vector comprising a betaretroviral pseudotype and a transgene encoding the CAR, wherein the viral vector is in an amount effective to transduce at least 3% of the starting population of γδ T cells, thereby producing the population of engineered γδ T cells expressing the CAR.

90. A method of producing a population of γδ T cells expressing a CAR and an armor protein, the method comprising:
(i) providing a starting population of γδ T cells; and
(ii) culturing the starting population of γδ T cells in the presence of IL-15 and a viral vector comprising a betaretroviral pseudotype, a first transgene encoding the CAR, and a second transgene encoding the armor protein, wherein the viral vector is in an amount effective to transduce at least 3% of the starting population of γδ T cells, thereby producing the population of engineered γδ T cells expressing the CAR and the armor protein.

91. The method of claim 90, wherein the armor protein is a cytokine.

92. The method of claim 91, wherein the cytokine is secreted.

93. The method of claim 92, wherein the cytokine is membrane-bound.

94. The method of any one of claims 91-93, wherein the cytokine is IL-15.

95. The method of any one of claims 89-94, wherein the starting population of γδ T cells lacks expression of ASCT-1 or ASCT-2.

96. The method of claim 95, wherein the starting population of γδ T cells lacks expression of ASCT-1 or ASCT-2.

97. The method of claim 89-96, wherein the population of engineered γδ T cells expresses ASCT-1 and/or ASCT-2.

98. The method of any one of claims 89-97, wherein the starting population of γδ T cells lacks functional expression of a VSV-G entry receptor.

99. The method of claim 98, wherein the VSV-G entry receptor is an LDL receptor.

100. The method of any one of claims 89-99, wherein the viral vector is cultured with the starting population of γδ T cells at an MOI no greater than 10.

101. The method of claim 100, wherein the MOI is no greater than 5.

102. The method of claim 101, wherein the MOI is from 1 to 5.

103. The method of any one of claims 73-102, wherein the betaretroviral pseudotype is BaEV

104. The method of any one of claims 73-102, wherein the betaretroviral pseudotype is RD114.

105. The method of any one of claims 73-104 , wherein the viral vector comprises a Retroviridae family viral vector backbone.

106. The method of claim 105, wherein the Retroviridae family viral vector backbone is a retroviral vector backbone.

107. The method of claim 106, wherein the retroviral vector backbone is a lentiviral backbone.

108. The method of claim 106, wherein the retroviral vector backbone is a gammaretroviral backbone.

109. The method of claim 106, wherein the retroviral vector backbone is an alpharetroviral backbone.

110. The method of any one of claims 73-109, wherein the engineered γδ T cells are Vδ1 T cells.

111. The method of any one of claims 73-109, wherein the engineered γδ T cells are Vδ2 T cells.

112. The method of any one of claims 73-109, wherein the engineered γδ T cells are non-Vδ1/Vδ2 T cells.

113. The method of any one of claims 58-112, wherein the CAR targets CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gpl20, HIV-1 envelope glycoprotein gp41, GD2, CD123, CD33, CD138, CD23, CD30, CD56, c-Met, mesothelin, GD3, HERV-K, IL-IIRalpha, kappa chain, lambda chain, CSPG4, ERBB2, EGFRvIII, VEGFR2, HER2-HER3 in combination, HER1-HER2 in combination, NY-ESO-1, SSX2, MAGE, MART-1, gp100, PSA, PSMA, PSCA, g9d2, or a combination thereof.

114. A population of engineered γδ T cells produced by the method of any one of claims 1-57.

115. The population of engineered γδ T cells of claim 114, wherein at least 10% of the population expresses a CAR.

116. The population of engineered γδ T cells of claim 115, wherein at least 10% of the population expresses a CAR and an armor protein.

117. The population of engineered γδ T cells of claim 115 or 116, wherein at least 50% of the population expresses the CAR.

118. The population of engineered γδ T cells of any one of claims 115-117, wherein at least 50% of the population expresses the CAR and the armor protein.

119. A population of γδ T cells expressing a CAR produced by the method of any one of claims 58-113.
